(19) United States
(12) Patent Application Publication
(10) Pub. No.: US 2022/0370747 A1
(43) Pub. Date: Nov. 24, 2022

(54) NASAL CANNULA AND SECUREMENT SYSTEM

(71) Applicant: Fisher & Paykel Healthcare Limited, Auckland (NZ)

(72) Inventors: Michael Paul RONAYNE, Auckland (NZ); Brad Michael HOWARTH, Auckland (NZ); Leon Tyler STANLEY, Auckland (NZ); Samuel Rollo Ross DAVIS, Auckland (NZ); Julio Derek MEECH, Auckland (NZ); Oscar Elliot James MCGERTY, Auckland (NZ); Hemanth PEMMARAJU, Auckland (NZ)

(21) Appl. No.: 17/763,626

(22) PCT Filed: Sep. 25, 2020
(86) PCT No.: PCT/IB2020/058973

§ 371 Date: Mar. 24, 2022

Related U.S. Application Data
(60) Provisional application No. 62/905,922, filed on Sep. 25, 2019.

(51) Int. Cl. A61M 16/06

(52) U.S. Cl. CPC A61M 16/0666; A61M 16/0605

(57) ABSTRACT

A patient interface for respiratory therapy includes a nasal interface having nasal prongs configured to contact nares of the user and form a seal. The patient interface also includes a securement portion having a pair of facial pads, each of the facial pads configured to engage the face of a user on opposing sides of the nasal interface. The facial pads are coupled to the bridging portions configured to urge the nasal interface toward the user's face to create or maintain sealing engagement between the nasal interface and the user's face. In some configurations, the securement portion and the nasal interface are detachably coupled to each other.

### BACKGROUND

### Field

The present disclosure generally relates to components for medical systems for conveying gases to and/or from a patient. In one particular aspect, the disclosure relates to patient interfaces that form a part of a breathing system.

### Description of the Related Art

In assisted breathing, respiratory gases are supplied to a patient through a patient interface via one or more flexible breathing tubes. The patient interface can be a nasal cannula, nasal mask, full face or oro-nasal mask, endotracheal tube, or other known types of interfaces. The gases expired by the patient may be channeled through a similar breathing tube to other equipment (valves, ventilators, pressure devices, or the like) or expelled to the patient's surroundings.

In medical applications, such as assisted breathing, the gases inhaled by a patient are preferably delivered close to body temperature (usually between 33° C. and 37° C.) and with a high relative humidity (commonly near saturation). In other medical applications, such as continuous positive airway pressure (CPAP) systems or positive pressure ventilations systems that provide patient's suffering obstructive sleep apnea (OSA) with positive pressure breathing gases, the breathing gases may be heated and/or humidified to varying levels to improve user comfort or supplied without heating or humidification.

It would be advantageous to provide a system for an alternative or improved operational positioning of the interface, such as a nasal cannula. Such an alternative or improved system may further assist with improved compliance of gas delivery treatment.

In the specification where reference has been made to patent specifications, other external documents, or other sources of information, this is generally for the purpose of providing a context for discussing the features of the disclosure. Unless specifically stated otherwise, reference to such external documents is not to be construed as an admission that such documents, or such sources of information, in any jurisdiction, are prior art, or form part of the common general knowledge in the art.

Further aspects and advantages of the present disclosure will become apparent from the ensuing description which is given by way of example only.

### SUMMARY

It would be advantageous to provide a system for an alternative or improved interface location or operational positioning of the interface, such as a nasal cannula, nasal mask, or oro-nasal mask. Such an alternative or improved system may further assist with improved compliance of gas delivery treatment.

An aspect of the present disclosure involves a patient interface including a nasal interface having a manifold and one or more prongs extending from the manifold. The interface also includes a securement portion, the securement portion including a body and a pair of bridging portions, each of which extending from the body. The nasal interface and the securement portion are detachably coupled to each other.

In some configurations, the one or more prongs are configured to be inserted into nares of a patient and form a seal.

In some configurations, the nasal interface is detachably attached to the securement portion at the body.

In some configurations, the patient interface further comprises a connection mechanism which detachably attaches the nasal interface with the securement portion.

In some configurations, the connection mechanism comprises a rigid portion.

In some configurations, the connection mechanism comprises one or more selected from a group consisting of: adhesive, hook or loop material, magnets, push-fit dome, snap-fitting, and a clip.

In some configurations, the connection mechanism comprises a clip attached to the body of the securement portion, wherein the clip is detachably attached to the nasal interface.

In some configurations, the patient interface further comprises one or more tubes configured to be connected to the manifold of the nasal interface.

In some configurations, the manifold includes one or more openings, wherein each of the openings is configured to receive the tube.

In some configurations, the one or more tubes are inspiratory or expiratory tubes.

In some configurations, the manifold comprises a lower portion with a curved profile.

In some configurations, the manifold comprises a patient contacting surface having an arcuate profile.

In some configurations, the manifold has a thinner region at a patient facing side.

In some configurations, the manifold has a thickened region under the one or more prongs.

In some configurations, each of the one or more prongs comprises a sealing region which is tapered toward a prong outlet.

In some configurations, each of the one or more prongs comprises a rounded corner at a lower end of the sealing region, wherein the taper of the sealing region starts from the rounded corner.

In some configurations, each of the one or more prongs further comprises an indicator positioned at the rounded corner, wherein the indicator is configured to indicate whether the one or more prongs are over-inserted into a user's nares when the patient interface is worn by the user.

In some configurations, the manifold comprises a thinned region between the prongs.

In some configurations, the manifold comprises a scalloped recess between the prongs.

In some configurations, the body of the securement portion comprises a lower edge having a curved profile.

In some configurations, the body of the securement portion comprises material stiffer than the pair of bridging portions.

In some configurations, the securement portion further comprises a pair of facial pads, wherein each of the pair of bridging portions extend from the body to each of the pair of facial pads.

In some configurations, the facial pads are configured to engage the face of a patient.

In some configurations, the patient interface further comprises a patch assembly coupled to each of the pair of facial pads for securing the pair of facial pads to the user.

In some configurations, the patch assembly comprises a hook or loop material.

In some configurations, the pair of bridging portions have a curved shape configured in use to urge the nasal interface toward a patient's face to create or maintain sealing engagement between the nasal interface and the user's nares.

In some configurations, the securement portion comprises a component which can be detachably attached to a respiratory tube or conduit.

In some configurations, the component comprises hook or loop material, magnets, or elastomeric band.

An aspect of the present disclosure involves a patient interface including a user interface portion and a securement portion. The securement portion includes a body and a pair of facial pads, each of the pair of facial pads configured to engage the face of a user on opposing sides of the body. The securement portion further includes a pair of bridging portions, each of the pair of bridging portions connecting the body and a respective one of the pair of facial pads. The user interface portion and the securement portion are detachably attached to each other.

In some configurations, the user interface portion is one selected from a group consisting of: a sealing cannula, a non-sealing cannula, a nasal mask, and an oronasal mask.

In some configurations, the user interface portion is detachably attached to the securement portion at a body of the securement portion. In some configurations, the patient interface further comprises a connection mechanism which detachably attaches the user interface portion with the securement portion.

In some configurations, the connection mechanism comprises a rigid portion.

In some configurations, the connection mechanism comprises one or more selected from a group consisting of: adhesive, hook or loop material, magnets, push-fit dome, snap-fitting, and a clip.

In some configurations, the connection mechanism comprises a clip attached to the body of the securement portion, wherein the clip is detachably attached to the user interface portion.

In some configurations, the patient interface further comprises one or more tubes configured to be connected to the manifold of the user interface portion.

In some configurations, the user interface portion comprises one or more openings, wherein each of the openings is configured to receive the tube.

In some configurations, the one or more tubes are inspiratory or expiratory tubes.

In some configurations, the user interface portion comprises a patient contacting surface having an arcuate profile.

In some configurations, the user interface has a thinner region at a patient facing side.

In some configurations, the patient interface further comprises a patch assembly coupled to each of the pair of facial pads for securing the facial pads to the user.

In some configurations, the patch assembly comprises a hook or loop material.

In some configurations, the pair of bridging portions have a curved shape configured in use to urge the user interface toward a patient's face to create or maintain sealing engagement between the user interface portion and the user's face.

In some configurations, wherein the pair of bridging portions are configured to roll in response to movement between the pair of facial pads and the body,

In some configurations, the bridging portions is configured to be stretched when the pair of facial pads are pulled.

In some configurations, the securement portion comprises a component which can be detachably attached to a respiratory tube or conduit.

In some configurations, the component comprises hook or loop material, magnets, or elastomeric band.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 shows an example of a system in which embodiments of the patient interface can be used.

FIG. 2 shows an example of a system in which embodiments of the patient interface can be used.

FIG. 3 is a front, top and side perspective view of a patient interface.

FIG. 4 is a front view of the patient interface of FIG. 3.

FIG. 5 is a rear view of the patient interface of FIG. 3.

FIG. 6 is a top view of the patient interface of FIG. 3.

FIG. 7 is a bottom view of the patient interface of FIG. 3.

FIG. 8 is a front, top and side perspective view of a patient interface.

FIG. 9 is a front view of the patient interface of FIG. 8.

FIG. 10 is a rear view of the patient interface of FIG. 8.

FIG. 11 is a top view of the patient interface of FIG. 8.

FIG. 12 is a bottom view of the patient interface of FIG. 8.

FIG. 13 is a front, top and side perspective view of a securement portion.

FIG. 14 is a front view of the securement portion of FIG. 13.

FIG. 15 is a rear view of the securement portion of FIG. 13.

FIG. 16 is a front, top and side perspective view of a securement portion.

FIG. 17 is a front view of the securement portion of FIG. 16.

FIG. 18 is a rear view of the securement portion of FIG. 16.

FIG. 19 is a front, top, and side perspective view of a clip.

FIG. 20 is a front view of the clip of FIG. 19.

FIG. 21 is a rear view of the clip of FIG. 19.

FIG. 22 is a top view of the clip of FIG. 19.

FIG. 23 is a front, top, and side perspective view of a clip.

FIG. 24 is a top view of the clip of FIG. 23.

FIG. 25 is a front view of the clip of FIG. 23.

FIG. 26 is a rear view of the clip of FIG. 23.

FIG. 27 is a front, top, and side perspective view of a nasal interface.

FIG. 28 is a front view of the nasal interface of FIG. 27.

FIG. 29 is a front, top, and side perspective view of a nasal interface.

FIG. 30 is a front view of the nasal interface of FIG. 29.

FIG. 31 is a top view of a nasal interface.

FIG. 32 illustrates a top view of a patient interface on the face of a user.

FIG. 33 is a rear, top and side perspective view of a nasal interface.

FIG. 34 illustrates a front view of a patient interface on the face of a user.

FIG. 35 is a cross-sectional view of a nasal interface

FIG. 36 is a cross-sectional view of the nasal interface of FIG. 35.

FIG. 37 is a partial top view of the nasal interface of FIG. 35.

FIG. 38 is a cross-sectional view of a nasal interface.

FIG. 39 is a cross-sectional view of a nasal interface.

FIG. 40 is a cross-sectional view of a nasal interface.

FIG. 41 is a cross-sectional view of a nasal interface.

FIG. 42 is a partial front view of a nasal interface.

FIG. 43 illustrates a schematic drawing of a prong of a nasal interface.

FIG. 44 illustrates a schematic drawing of a portion of a nasal interface.

FIG. 45 illustrates a front, top, and side perspective view of a patient interface and interface tubes.

FIG. 46 illustrates a perspective view of the interface tubes of FIG. 45.

FIG. 47 illustrates a perspective view of the interface tubes of FIG. 45.

FIG. 48 illustrates a bottom view of a securement portion.

FIG. 49 illustrates a portion of the securement portion of FIG. 48.

FIG. 50 illustrates a patient interface applied on the face of a user.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made or directions relative to an orientation of the device as used with the user in an upright position. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion or relative to an orientation of the device as used with the user in an upright position.

### Respiratory System

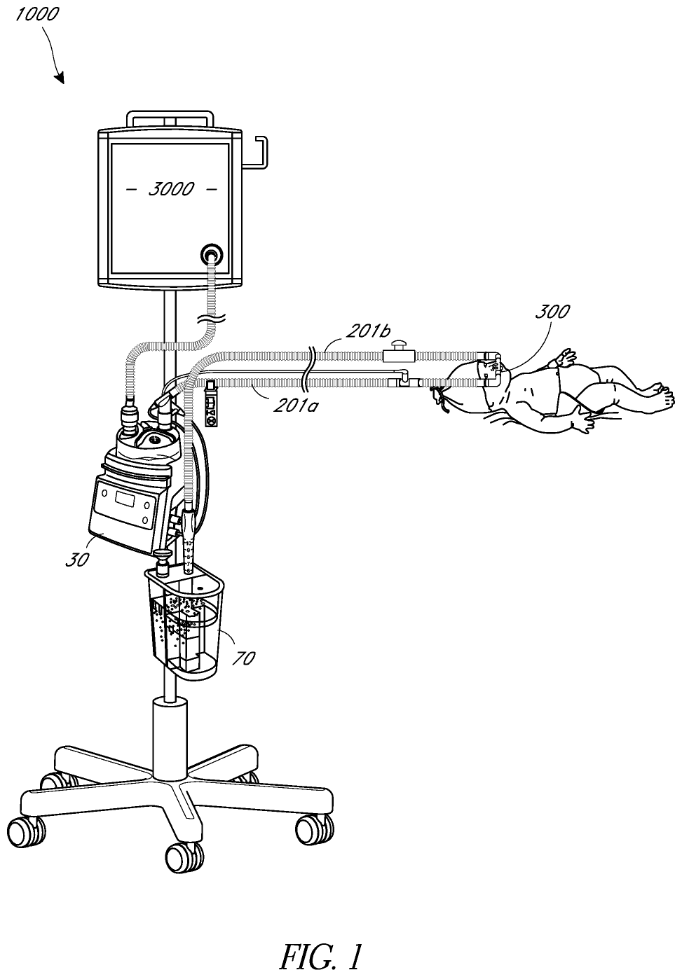

FIG. 1 shows an example respiratory system 1000 in which embodiments of a patient interface 300 can be used. In the illustrated arrangement, the patient interface 300 receives an inspiratory flow of gases via an inspiratory conduit 201a, and a flow of the expiratory gases is directed from the interface 100 via an expiratory conduit 201b to a resistance device, which in the illustrated arrangement is a bubbler device 70. An optional humidifier system 30 is provided to humidify the inspiratory flow of gases. The humidifier system 30 typically includes a chamber sitting atop a heater base, the chamber of which is fed with a source of gases flow from, for example, a hospital or other supply source 3000. The humidified inspiratory flow of gases is delivered to the airway of the patient by inspiratory conduit 201a and the patient interface 300. Excess and expired gases are evacuated from the patient interface 300 by the expiratory conduit 201b. The resistance device 70 provides resistance to the expiratory flow of the gases exiting the system 1000 to the atmosphere to provide a desirable peak end expiratory pressure (PEEP). One of skill in the art will understand that such a system may include additional and/or replacement components as are known in the art. In some embodiments, the patient interface 300 is a nasal cannula. In other embodiments, the patient interface 300 can be a mask. For example, the patient interface 300 may be a nasal mask, an oro-nasal mask, an oral mask or a full-face mask. In some embodiments, the resistance device and/or the humidifier may be integrated into the supply source 3000. Although a water-based resistance device is shown in FIG. 1, it should be appreciated by one of skill that the resistance device may be any other mechanical or electrical resistance device as is known in the art.

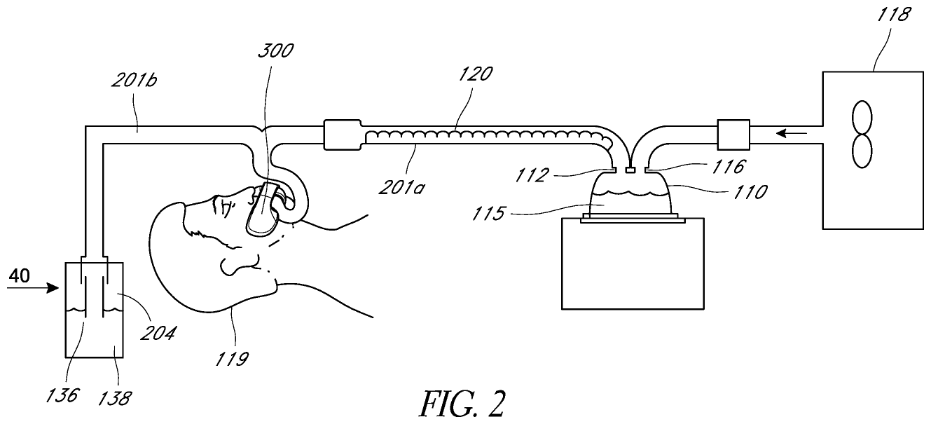

Referring now to FIG. 2, another example of a respiratory system including a bubbler device and humidifier is depicted. A humidified Positive End Expiratory Pressure (PEEP) system is shown in which a patient 119 is receiving humidified and pressurised gases through the patient interface 128 connected to an inspiratory or inhalatory conduit 201a. It should be understood that the present invention, however, is not limited to the delivery of PEEP gases but is also applicable to other types of gases delivery systems and may not necessarily involve humidification. Inspiratory conduit 201a is connected to the outlet 112 of a humidification chamber 110, which contains a volume of water 115. Inspiratory conduit 201a may contain heating means or heater wires 120 that heat the walls of the conduit to ensure a constant humidity profile along the conduit and therefore reduce condensation of humidified gases within the conduit. As the volume of water 115 within humidification chamber 110 is heated, water vapour begins to fill the volume of the chamber above the water's surface and is passed out of the humidification chamber 110 outlet 112 with the flow of gases (for example air) provided from a gases supply means or blower 118 which enters the chamber 110 through inlet 116.

The humidified gases pass through the inspiratory conduit 201a to an interface 128 being worn by the patient 119. The excess gases then flow through an expiratory or exhalatory conduit 201b to a pressure regulator 40.

In some embodiments, the pressure regulator 40 takes the form of discharging the flow of exhalatory gases into a chamber 204 containing a column of water 138. The gases flowing through the expiratory conduit 201b are discharged into the body of water 138 from a short conduit 136 which extends from the expiratory conduit 201b into the chamber 204. This results in a bubbling effect, whereby the gases eventually exit the chamber 204 via the outlet port, which can also be used to initially fill the chamber 204 with water. The outlet port includes shielding to prevents liquid aerosols created by the vigorous bubbling on the surface of the water from being expelled. It will be appreciated that the short conduit 136, could equally be integrated into the end of the expiratory conduit 201b.

### Patient Interface

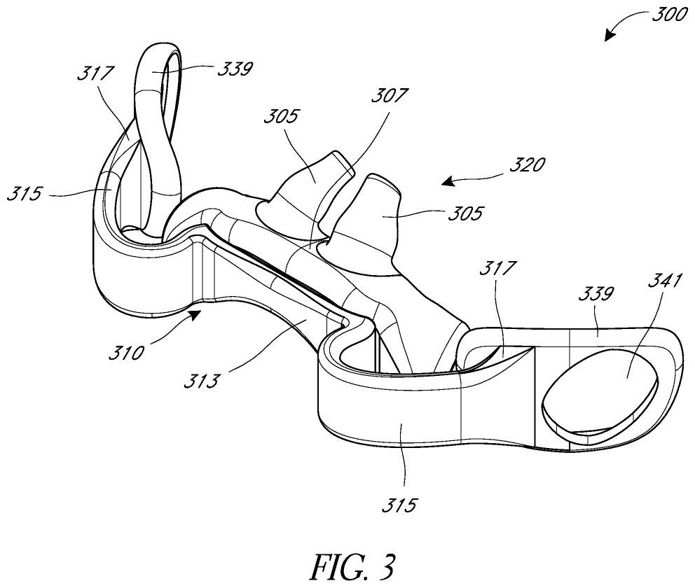

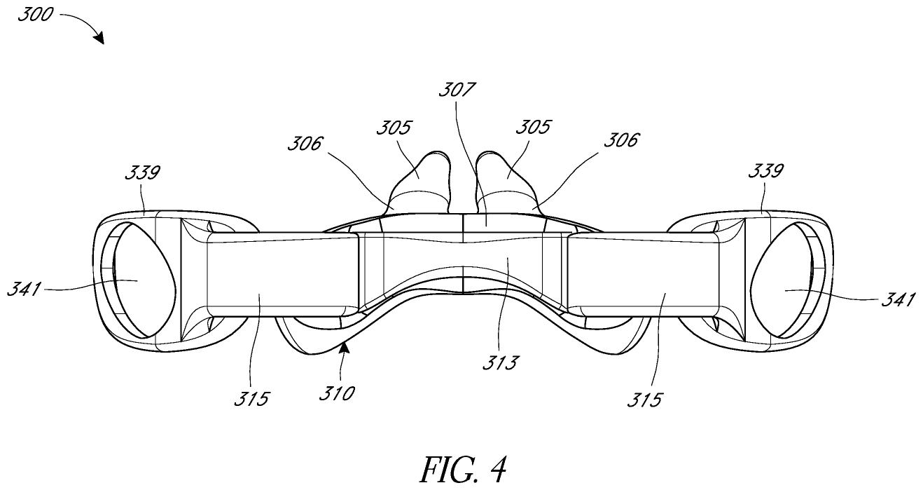

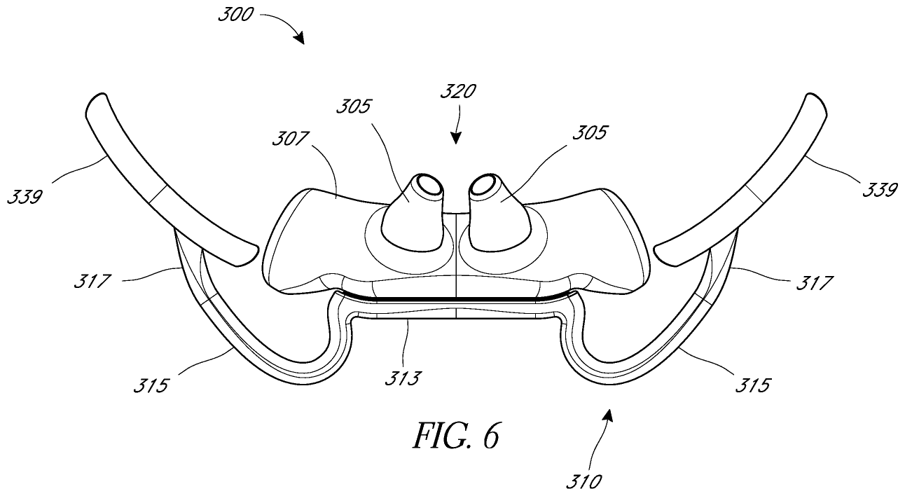

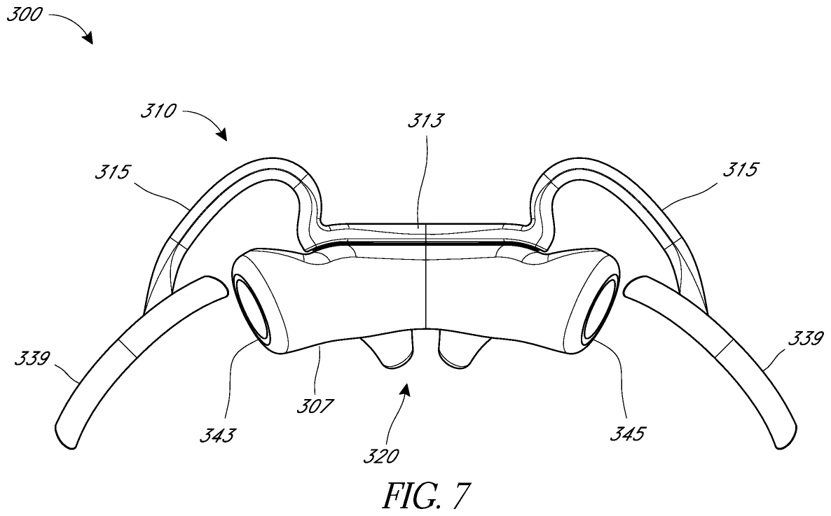

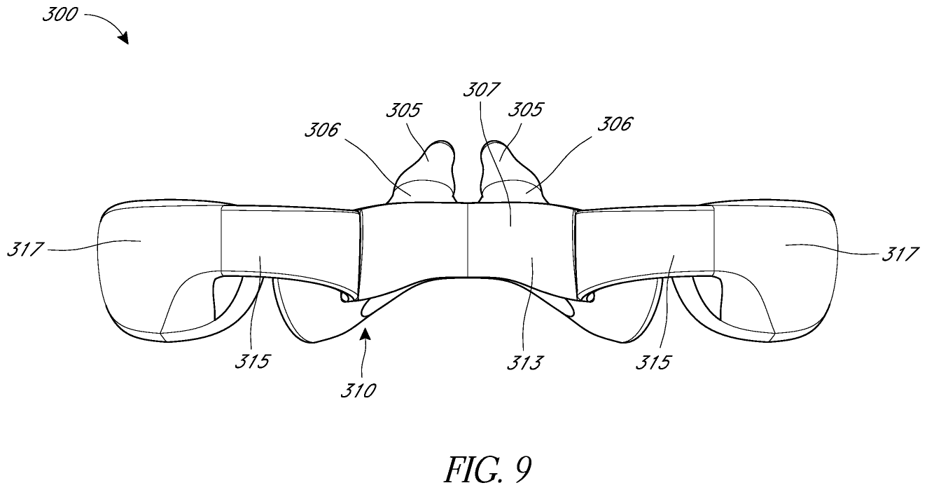

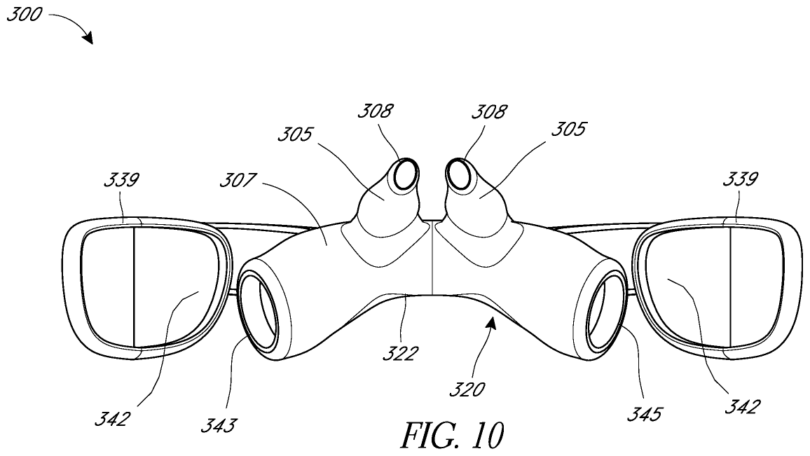

FIGS. 3-11 illustrate an embodiment of the patient interface 300. As shown in FIGS. 3-11, the patient interface 300 includes a nasal cannula portion 320 and a securement portion 310. The nasal cannula portion 320 may have a manifold 307 and at least one nasal prong 305 extending from the manifold 307, which can form a seal with nares of a user's nose and allow gas flow from and to the user. In some embodiments, the nasal prong 305 may not form a seal with nares of a user's nose. The securement portion 310 may include one or more feet, cheek supports or facial pads 339 for the securement or supporting of the patient interface 300 relative to the user, and one or more side arms or bridging portions 315 each extending from a body 313 to the facial pad 339. The securement portion 310 may facilitate securement of the nasal cannula 300 to the user's face and thereby help the nasal cannula portion 320 to form and maintain a seal with nares.

Even though the nasal cannula portion 320 is coupled with the securement portion 310 in the illustrated embodiments, the nasal cannula portion 320 may be used without the securement portion 310. For example, the nasal cannula portion 320 may be secured on the user's face with other securement means, such as a headgear or chinstrap.

In some embodiments, the nasal cannula portion 320 and the securement portion 310 may be detachably coupled to each other, for example using mechanisms to be described in further detail herein, such that a user or a clinician can swap between nasal cannulas with different sizes for better sizing of the prongs when fitting a nasal cannula to a patient, or even swap between different type of interfaces (e.g. between a mask and a cannula) while reusing the same securement portion during treatment. In some occasions, a mask and a cannula need to be used alternately during treatment to reduce or prevent skin irritation or damage, and the interface could be swapped while maintaining the securement portion connected to a headgear, a chinstrap, or the face of the user. Further, the securement portion 310 would be able to be swapped between different sizes without removing a nasal cannula or a mask from the user's face. For example, an infant's face may grow and a larger securement portion may be needed, while nares of the infant's nose may not grow much, such that the same nasal cannula needs to be used. Since the interface (e.g. nasal cannula portion 320) and the securement portion 310 can be sized individually and combined, the patient interface may fit the user's face better, allowing a better seal and preventing skin damage. Further, detachable coupling of the nasal cannula portion 320 and the securement portion 310 may be advantageous for cleaning and maintenance of the patient and/or the interface. However, in some embodiments, the nasal cannula portion 320 and the securement portion 310 may be permanently attached to each other, for example for better structural integrity or the ease of manufacturing.

In some embodiments, the nasal cannula portion 320 and the securement portion 310 may be constructed from different materials. For example, the nasal cannula portion 320 may be constructed from a stiffer material to prevent kinking or collapsing of the prongs 305 and/or manifold 307, while the securement portion 310 may be constructed from a softer material to better absorb the movement of the user's face and isolate it from the nasal cannula portion 320.

### Securement Portion

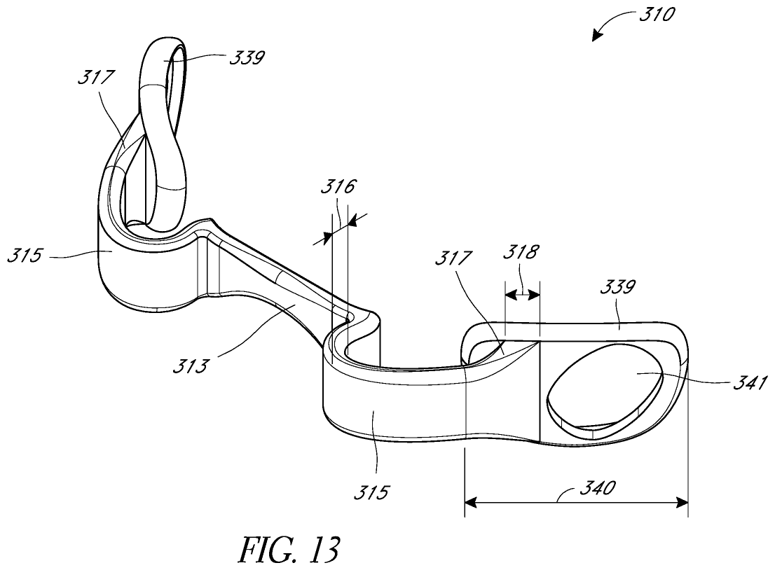

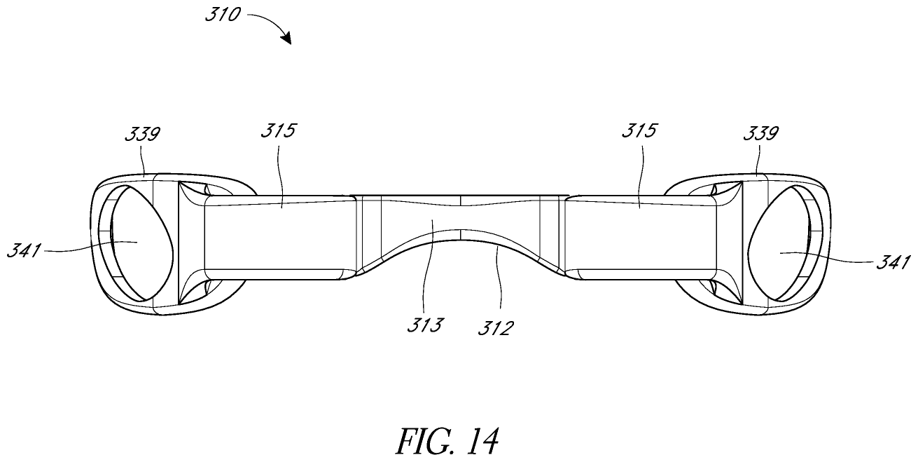

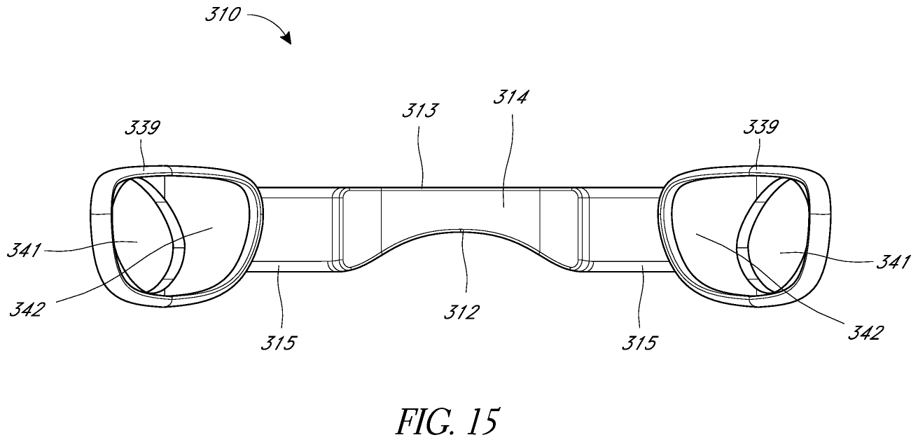

The securement portion 310 may include the body 313 and the bridging portions 315, each extending from the body 313 to the facial pad 339. FIGS. 3-7 illustrate an embodiment of the securement portion 310 coupled with the nasal cannula portion 320, and this embodiment of the securement portion 310 is separately illustrated in FIGS. 13-15.

As illustrated in FIGS. 13-15, the body 313 may have a generally planar shape. In some embodiments, the body 313 may have a generally rectangular shape. In the illustrated embodiment, the body 313 has a lower edge 312. In some embodiments, at least a portion of the lower edge 312 is contoured upward, as described in further detail elsewhere herein. In some embodiments, at least a portion of the lower edge 312 can be at least substantially straight. In the illustrated embodiment, the body 313 has a user-facing side 314. In some embodiments, the user-facing side 314 can receive or attach to a clip 330 which can connect the securement portion 310 with the nasal cannula portion 320. The clip 330 is described in further detail elsewhere herein. The user-facing side 314 may be contoured such that it conforms to the contour of the clip 330 and/or the nasal cannula portion 320.

In some embodiments, the body 313 is permanently or semi-permanently connected with the bridging portions 315. In some embodiments, the body 313 and the bridging portions 315 are integrally formed in a single, unitary piece. In some embodiments, the body 313, the bridging portions 315, and the facial pads 339 may be integrally formed in a single, unitary piece. For example, the body 313, the bridging portions 315 and the facial pads 339 may be molded as a single piece from a single material, or co-molded or over-molded as a single piece from different materials. In some cases, the ease of manufacturing may be improved if the body 313 and the bridging portions 315 are individually formed and subsequently assembled or coupled.

In the illustrated arrangement, each of the facial pads 339 includes a base or connecting portion 317 that is coupled directly to the respective bridging portion 315. In some configurations, the base or connecting portion 317 is configured to provide support to a face-contacting portion of the facial pad 339.

As described herein, the facial pads (e.g., the facial pads 339) can rest directly or indirectly on a user's face when the patient interface (e.g., the patient interface 300) is worn by the user. Accordingly, the bridging portions (e.g., the bridging portion 315), which are connected to the facial pads, may be moved or deflected when the user's face moves. For example, when a portion of the user's face on which the facial pads rest moves, such external force or movement may be conducted to the bridging portion, then to the body (e.g., the body 313), and eventually to the nasal cannula (e.g., the nasal cannula portion 320). In some instances, the bridging portions themselves may move or deflect when an external force is exerted on them. For example, the user of the patient interface may lay on the bridging portions, exerting force to the bridging portions, and thus to the nasal cannula. Accordingly, it is desirable for the bridging portion to be constructed such that the nasal cannula can maintain its seal with the user's nares even when the user's face and/or the facial pads move.

To apply the patient interface 300 to the user, the nasal cannula portion 320 may be first positioned into nares of the user. With the bridging portions 315 and the facial pads 339 in a relaxed position, the facial pads 339 may be spaced away from the user's face. With the nasal cannula portion 320 positioned in the user's nares, the facial pads 339 may be moved from the relaxed position toward the face of the user until the facial pads 339 contact or rest upon the user's face. The movement of the facial pads 339 from the relaxed position toward the face of the user will likely cause movement (e.g., flexing or extension) of the bridging portions 315. The facial pads 339 can be secured to the user's face directly or indirectly by an external force, such as using an adhesive connection, a headgear arrangement, a hook or loop connection as discussed above, or another suitable support structure, such as any of those described herein.

When the facial pads 339 are positioned on the face of the user, the bridging portions 315 preferably generate a force tending to apply upward pressure to the body 313 and thus to the nasal cannula portion 320, which helps create or maintain a seal between the nasal cannula portion 320 and the user. The force is generated as a result of deformation of the bridging portions 315 from their relaxed configurations and the resulting material strain tending to revert the bridging portions 315 back towards their relaxed configurations.

Advantageously, if the relaxed configuration of the bridging portion 315 spaces the respective facial pad 339 away from the user's face, the user or a clinician is provided with the freedom to fit and position the facial pads 339 on the user's face as desired. Such an arrangement facilitates adjustment or customization of the fit of the patient interface 300. For example, extending or deforming the bridging portions 315 in an outward and partially upward direction before securing the facial pads 339 increases the upward force acting on the body 313 and the nasal cannula portion 320 in comparison to simply move the facial pads 339 straight towards the user's face. The bridging portions 315 may be constructed to limit upward force to an acceptable level to inhibit or prevent the user or the clinician from overtightening the patient interface 300 by way of overextension of the bridging portions 315, while retaining some adjustability to reduce, eliminate or otherwise address leaks that may occur.

In some configurations, the deformable configuration of the bridging portions 315 provides some degree of de-coupling of the facial pads 339 from the nasal cannula portion 320. For example, deformation of the bridging portions 315 may allow cheek movement of the user to have a reduced or minimal effect on the upward pressure between the nasal cannula portion 320 and the bridging portions 315 in comparison to a straight or substantially straight bridging portion. Preferably, the deformation of the bridging portions 315 at least reduces or possibly eliminates disruption of the seal between the nasal cannula portion 320 and the user's face as a result of cheek movements of the user's face.

Some or all of these advantages may be provided by one or more of the shape of the bridging portions 315, the orientation of the bridging portions 315 or the material(s) used to construct the bridging portions 315. In some embodiments, the bridging portions 315 are each shaped such that its curved geometry facilitates the flexibility of the bridging portions 315. The bridging portions 315 may have a relatively gentle curve/radius. In some such configurations, the bridging portions 315 roll in and out, in contrast to folding like a hinge, during movement. The bridging portion 315 may define a curved shape configured to roll or hinge in response to movement between the facial pad 339 and the nasal cannula portion 320. The curved profile, cross-sectional geometry and/or material of the bridging portion encourages the de-coupling of the movement of the nasal cannula portion 320 from the movement of the facial pad 339, enabling the bridging portions 315 to absorb forces exerted on them, for example caused by the movement of the patient's cheek (in any direction). In addition, the flexibility of the bridging portions 315 may provide the user with the ability to stretch the arms on application, generating a force resisting prong movement out of the nares to help maintain a seal. This may also enable the user to adjust the applied force depending on the patient's facial geometry and/or size. The flexibility of the bridging portions 315 preferably allows the bridging portions 315 to absorb forces exerted on them, for example caused by the movement of the user's cheek. Accordingly, as described above, only some of the force exerted on the facial pads 339 and, thus, the bridging portions 315 is transferred to the nasal cannula portion 320. Advantageously, such an arrangement assists in maintaining the seal between the nasal cannula portion 320 and the user's face during movement of the user's cheeks. Various configurations of such bridging portions are further described in U.S. Provisional Application No. 62/812,823, the contents of which are incorporated herein by reference.

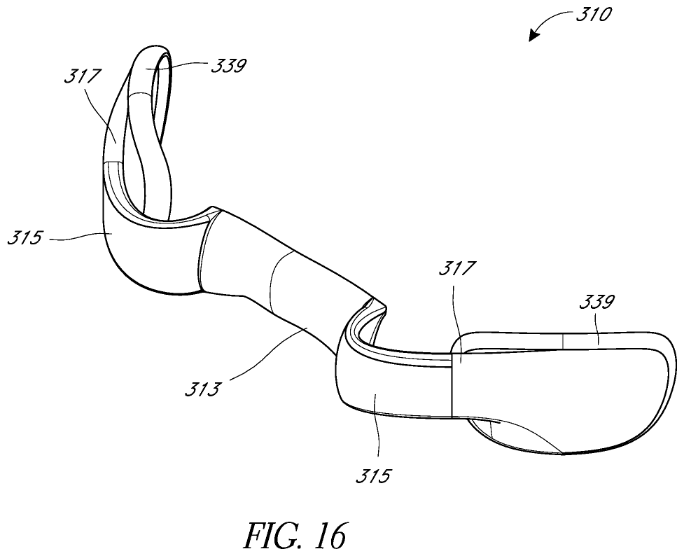

As illustrated in, for example, FIG. 13 and FIG. 16, the bridging portions 315 may be provided as elongate arms shaped to have or define a non-linear or curved path between a first end at a junction between the body 313 and the bridging portion 315 and a second end at a junction between the bridging portion 315 and the base 317 of the associated facial pad 339. Following the curved path from the first end to the second end, the path preferably defines at least a forwardly-extending portion followed by at least rearwardly-extending portion relative to the overall interface 300 wherein a forward direction is toward the front of the interface or away from the user and the rearward direction is toward the rear of the interface or toward the user.

Such an arrangement increases the effective length of the bridging portion 315, in a space-efficient manner, thereby lessening the bulkiness of the user interface 300 and improving the comfort of the user. A greater effective length of the bridging portions 315 can provide the bridging portions 315 a greater range of movement or adjustment, such that the user interface 300 can be applied to users with a wider range of face sizes. In addition or in the alternative, a greater effective length provides the bridging portions 315 with a flatter force-extension curve, which may inhibit or prevent the nasal cannula portion 320 from exerting too much force on the user's nares even when the bridging portions 315 are significantly stretched while in use. A flatter force-extension curve provides a more constant sealing force on a patient's face during cheek movement.

As shown in FIG. 13 and FIG. 16, the bridging portions 315 may have a relatively gentle curve/radius. In some such configurations, the bridging portions 315 roll in and out, in contrast to folding like a hinge, during movement. In some embodiments, the bridging portions 315 have a radius of curvature of between about 0.5 cm-30 cm, inclusive, including any value within this range, such as about 0.75 cm, 1 cm, 2 cm, 5 cm, 10 cm, 15 cm, 20 cm, 25 cm or 30 cm, at any location along the length of the bridging portion 315. In some embodiments, the radius of curvature can be greater. In some embodiments, each of the bridging portions 315 may have substantially constant or smoothly varying curvature throughout its length, such that the bridging portions 315 do not include an abruptly bent or folded point. In some embodiments, each of the bridging portions 315 may have at least partially an elliptical shape, a circular shape, a hook shape, a U-shape, or an S-shape as viewed from above or below the interface 300. In some embodiments, the bridging portions 230 may have an at least partially circular or elliptical cross-section.

In some embodiments, the bridging portions 315 are made of a flexible material, such that the bridging portions 315 are flexible or deformable, as described above. In some embodiments, the bridging portions 315 are made of the same material as the body 313 in an assembled or unitary construction. In some embodiments, the bridging portions 315 may be made of one or more elastomer materials, such as silicone, rubber, polyethylene, etc. In some embodiments, the bridging portions 315 may be made of a single material. In some embodiments, the bridging portions 315 may be made of two or more materials, such that a region of the bridging portion 315 has a different flexibility relative to another region.

In some embodiments, each of the bridging portions 315 may be shaped such that its geometry contributes to the flexibility. In some embodiments, the bridging portions 315 may have a constant or substantially constant cross-sectional area throughout their length. In some embodiments, the bridging portions 315 may have a varying cross-sectional area along their length, such that, for example a region of the bridging portion 315 has greater cross-sectional area and, assuming the same material, is stiffer than another region.

In some configurations, the base or connecting portion 317 may be flexible, such that the bridging portions 315 can be substantially isolated from the movement of the facial pads 339. FIG. 13 illustrates a thickness 318 of the base of connecting portion 317 and a thickness 316 of the bridging portion 315. In some configurations, the thickness 318 of the connecting portion 317 can be substantially similar to the thickness 316 of the bridging portion 315. In other words, the connecting portion 317 may be configured to permit flexibility or rotational movement of the facial pad 339 relative to the connecting portion 317 and, thus, the bridging portion 315. For example, the base or connecting portion 317 may include a flexible material or a hinge, a thinned portion or a cut-out for greater flexibility. In some embodiments, the thickness 318 of each of the base or connecting portion 317 may be less than 50%, 40%, 30%, 20%, 10%, or 5% of a width 340 of the corresponding facial pad 339. In some embodiments, the thickness 318 of each of the base or connecting portion 317 may be less than 100%, 110%, 125%, 150%, 160%, 170%, 180%, 190%, 200%, or 300% of the thickness 316 of the corresponding bridging portion 315.

In some embodiments, as illustrated in FIG. 13-15, each of the bridging portions 315 is attached to the body 313 separately, thereby allowing movement independent from each other. For example, when one cheek and thus one facial pad 339 and one bridging portion 315 are moved (e.g., when the user is lying on his side or when the user's face is squeezed), the other bridging portion 315 may not move, and a sufficient seal may be maintained.

To further isolate the movement of one facial pad 339 and one bridging portion 315 from the body 313, and thus the other facial pad 339 and the other bridging portion 315, a junction or connection point between the bridging portion 315 and the body 313 may have greater flexibility. In some embodiments, the junction or connection point includes a hinge or a cut-out for greater flexibility. Further, to prevent the movement of one facial pad 339 from causing the excessive movement of the bridging portion 315 and the rest of the securement portion 320, a connection point or junction (e.g. the base or connecting portion 317) may have greater flexibility. In some embodiments, the junction or connection point includes a hinge or a cut-out for greater flexibility.

The backing or facial pad 339 can receive and support one or more layers of a patch (e.g. a user interface patch or a dermal patch) or adhesives on a user-facing surface 342, such that the backing or facial pad 339 can rest on the user's face without directly contacting the user's skin when the user interface is secured on the user's face. The patient-facing surface 342 of the facial pad 339 can be initially provided without a user interface patch or a dermal patch, and the surface 342 can receive or retain a patient interface patch or a dermal patch. Such a patient interface patch may be connected to the surface 342 by an adhesive or other suitable connection as is known in the art (such as by hook-and-loop fastener, ultrasonic welding, and/or co-molding or overmolding). Once the patch is in position, it is ready to be connected to or receive a dermal patch. Alternatively, the patient interface patch may be assembled to the surface 342 of the facial pad 339 in the same operation as molding the facial pad 339.

In the illustrated embodiment of FIG. 13-15, each of the backing or facial pads 339 includes an opening 341 formed through the thickness of the backing or facial pads 339. When the user interface patch is applied to the user-side surface 342, the opening 341 may expose the user interface patch from the front side opposite the user-side. Accordingly, if the patient interface patch is connected to the surface 342 using a double-sided adhesive or a double-sided hook-and-loop fastener, such an adhesive or hook-and-loop fastener will be exposed from the front side opposite the user-side. In some configurations, such an exposed adhesive or hook-and-loop fastener through the opening 341 may be used as a tube fastener or otherwise used to organize tubes or conduits (e.g. the interface tubes to be connected to the inspiratory conduit 201a, the expiratory conduit 201b) as further described elsewhere herein.

In some arrangements, the backing or facial pad 339 can be connected to a headgear and/or a chinstrap for the securement of the nasal cannula 300 can be otherwise connected to a headgear and/or a chinstrap, which are common components known in the art.

The backing or facial pads 339 may be pre-formed to be of a contour that is substantially curved to fit a user's face, cheek or upper lip region. The base or connecting portion 317 of the bridging portions 315 may be also contoured to accommodate the contour of the facial pads 339. The facial pads 339 may be anatomically shaped with a distribution and scale of curvature that reflects the facial geometry of the intended user. The anatomical shape of the facial pads 339 can give the interface a positive engagement with a user's face at a predetermined position where the contour of the facial pads 339 matches the user's facial contour. Pre-shaping or contouring the facial pads 339 to the user's facial features reduces the pressure applied to the user's face by any retention mechanism (adhesive tape, headgear or other means). This reduces the likelihood of pressure sores, head forming, or other injury. The positive engagement promoted by the anatomical shape of the facial pads 339 increases the stability of the nasal cannula 300 and therefore improves comfort and efficacy of the treatment being administered.

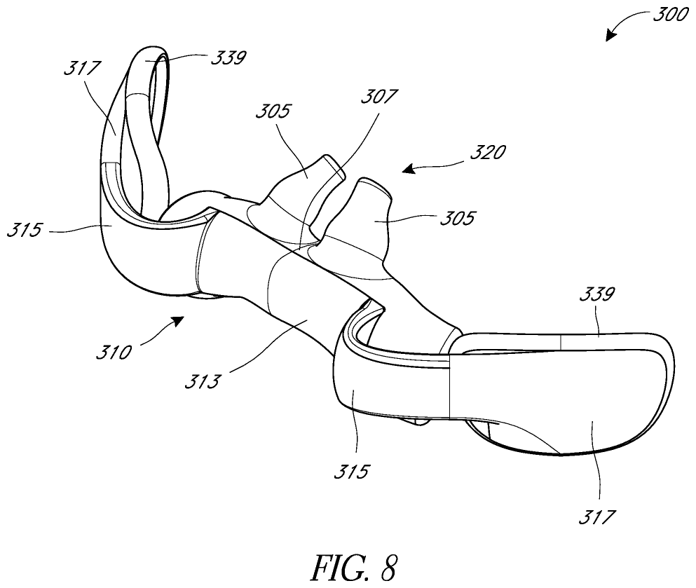

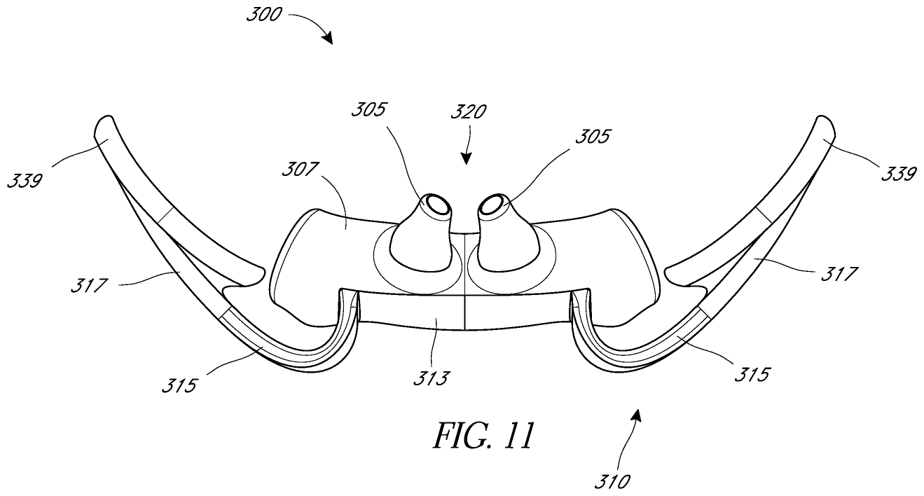

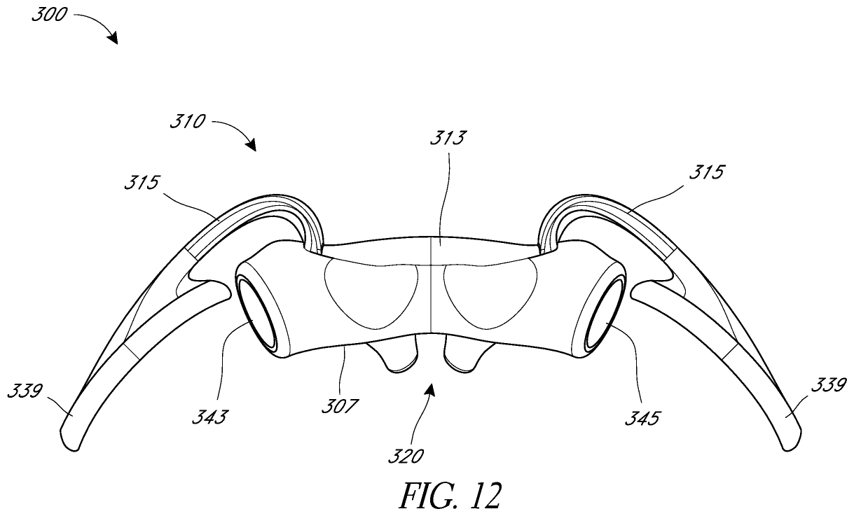

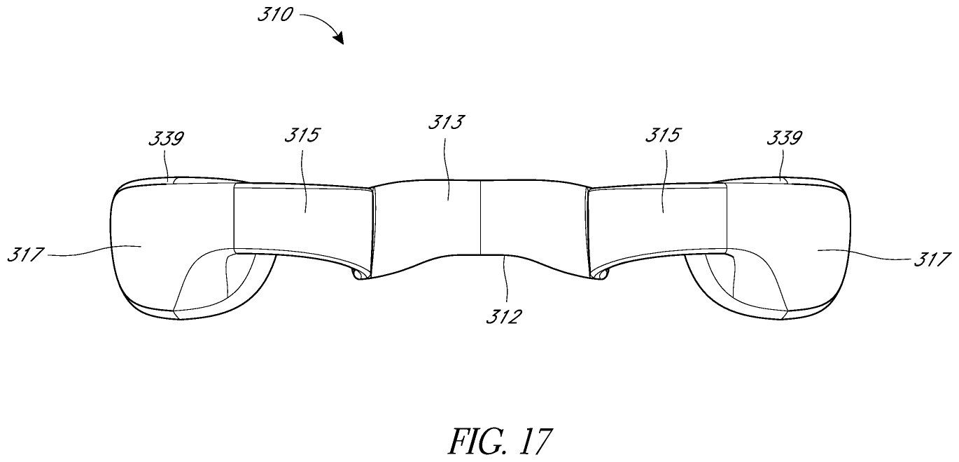

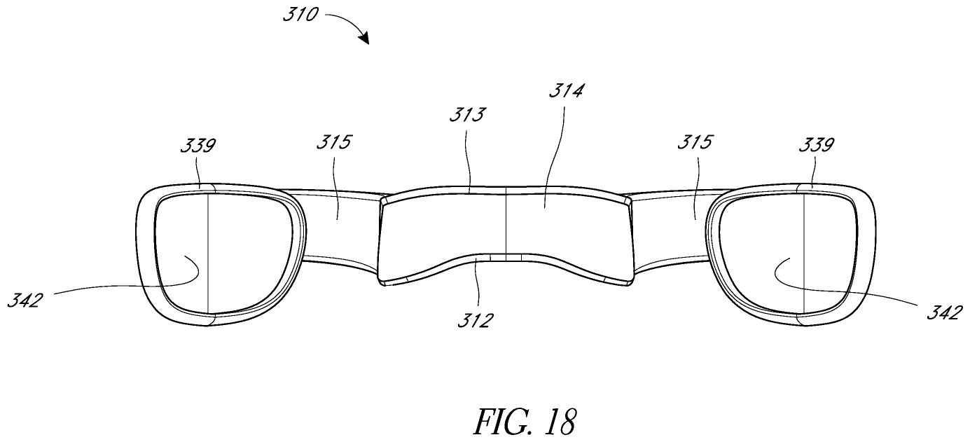

FIGS. 16-18 illustrate another embodiment of the securement portion 310. The illustrated embodiment of the securement portion 310 in FIGS. 16-18 can be similar with the illustrated embodiment of FIGS. 13-15, other than the appearance and features noted below. For example, the securement portion 310 of FIGS. 16-18 can be coupled with the nasal cannula portion 320 to form a patient interface 300, as shown in FIGS. 8-12. In the illustrated embodiment, the body 313 has a contour which is generally continuous and conforming to the contour of the nasal cannula portion 320. In the illustrated embodiment, the facial pads 339 do not include any openings. In the illustrated embodiment, the base 317 is generally triangular in shape as viewed from above (see, e.g., FIGS. 11-12).

### Coupling of Nasal Cannula Portion and Securement Portion

As discussed elsewhere herein, the nasal cannula portion 320 and the securement portion 310 may be detachably coupled to each other. Any suitable detachable connection mechanism known in the art may be used for the connection between the nasal cannula portion 320 and the securement portion 310. For example, adhesive or glue attachment, Velcro® or hook or loop attachment, slots and protrusions, magnets and ferromagnetic materials, snap-fit or push-fit attachment (e.g. snap domes or buttons) or any combination of these can be used, and the nasal cannula portion 320 and the securement portion 310 may include respective structures for the detachable connection. In some embodiments, the securement portion 310 may include a rigid annular frame which can clip in place around at least a portion of the nasal cannula portion 320.

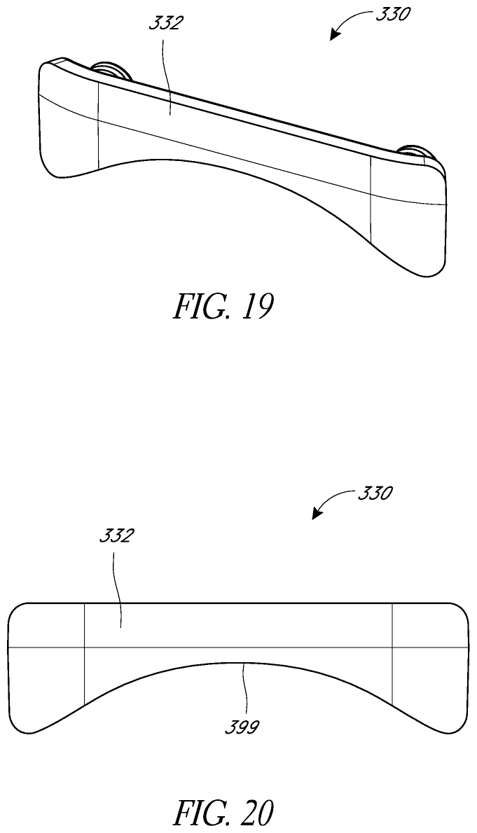

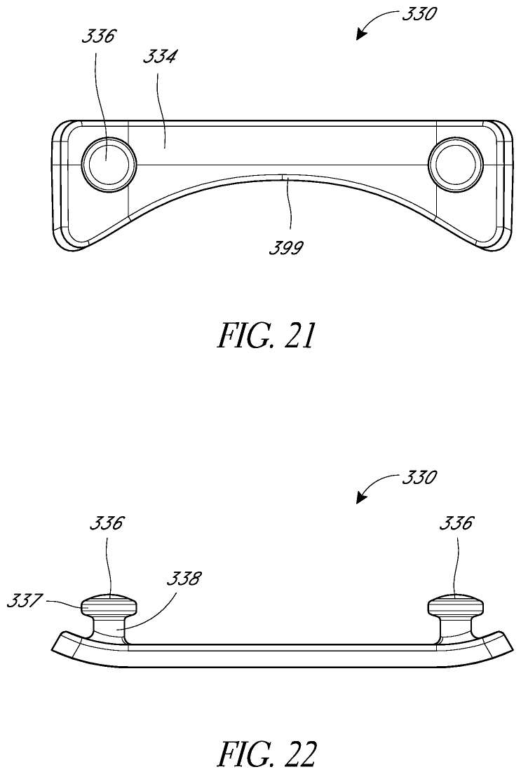

In some embodiments, the patient interface 300 may include the clip 330 which can be attached to the securement portion 310 and detachably connect the securement portion 310 with the nasal cannula portion 320. FIGS. 19-22 illustrate an embodiment of the clip 330 having push-fit domes 336. The clip 330 may have a user-facing side 334 and a forward-facing side 332 opposite the user-facing side 334.

The forward-facing side 332 may be attached to the body 313 of the securement portion 310, for example at the user-facing side 314. The forward-facing side 332 can be attached to the body 313 using any suitable mechanisms. The clip 330 may be permanently attached to the body 313 of the securement portion 310, for example overmoulded or welded, such that it can be removed with the securement portion 310 when the securement portion 310 is detached from the nasal cannula portion 320. In some embodiments, the clip 330 is integrally molded or manufactured as a single piece with the body 313. In some embodiments, the clip 330 is detachably attached with the body 313, for example with an adhesive or hook or loop fastener. In some embodiments, the clip 330 may be permanently attached to the nasal cannula portion 320, and can be detachably attached to the securement portion 310.

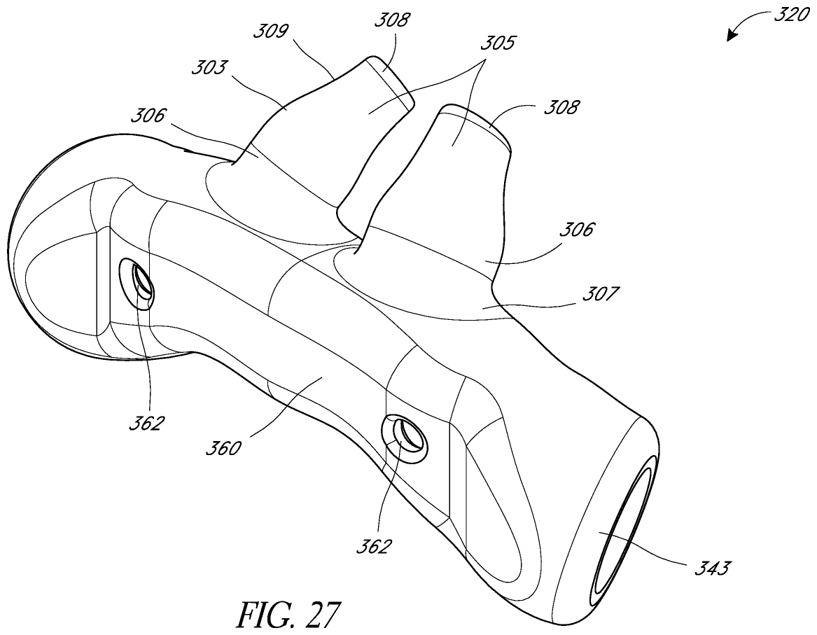

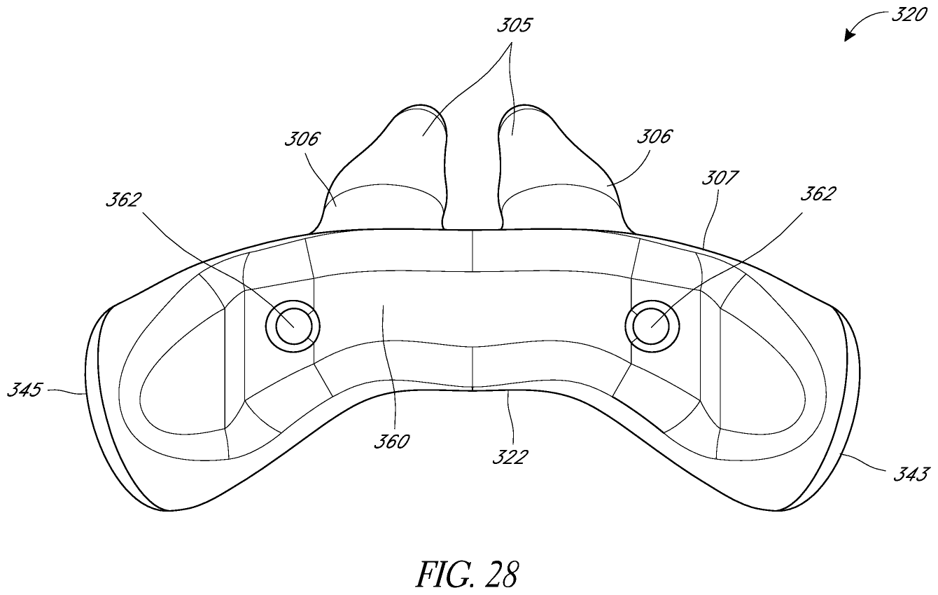

In the illustrated embodiment, the user-facing side 334 of the clip 330 includes push-fit domes 336 protruding from the clip 330. Each of the push-fit domes 336 includes a stem 338 and an enlarged head 337. Even though the illustrated embodiment of the clip 330 includes two push-fit domes 336, the clip 330 may include one, three, four or more push-fit domes 336. When the securement portion 310 couples with the nasal cannula portion 320, the user-facing side 334 of the clip will be attached to a clip-receiving region 360 of the nasal cannula portion 320 shown in FIGS. 27-28. The clip-receiving region 360 may further include one or more push-fit holes 362, which can reversibly receive the push-fit domes 336, such that the clip 330 can be coupled with the nasal cannula portion 360 by pushing the domes 336 into the holes 362. As shown in FIG. 28, the holes 362 may be laterally spaced away from each other further than the spacing between the prongs 305, such that the prongs 305 are laterally positioned between the holes 362. However, the holes 362 may be positioned or arranged differently depending on the clip 330. In some embodiments, the user-facing side 334 of the clip 330 can include components for other detachable coupling mechanisms discussed herein, in place of or in addition to the push-fit domes 336, and the clip-receiving region 360 of the nasal cannula portion 320 can include corresponding components for detachable coupling with the clip 330 and the nasal cannula portion 320.

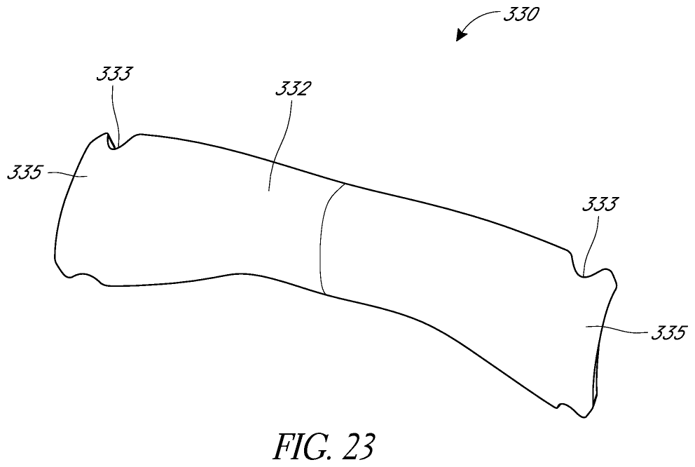

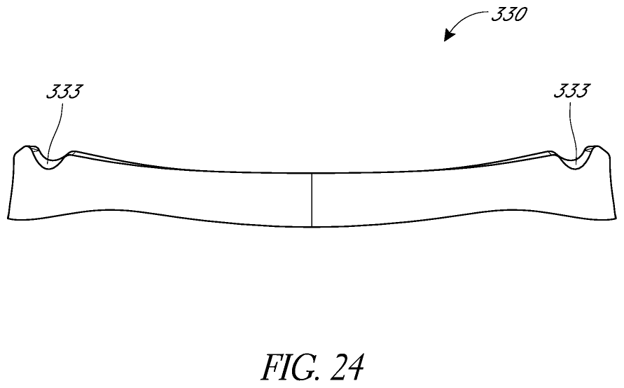

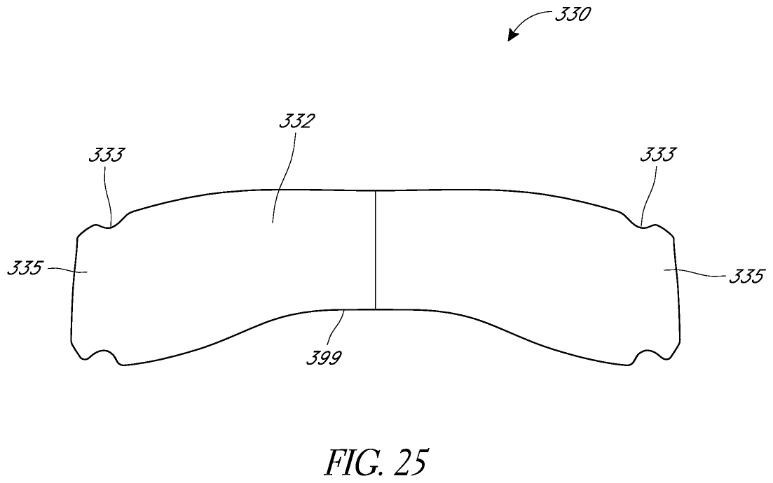

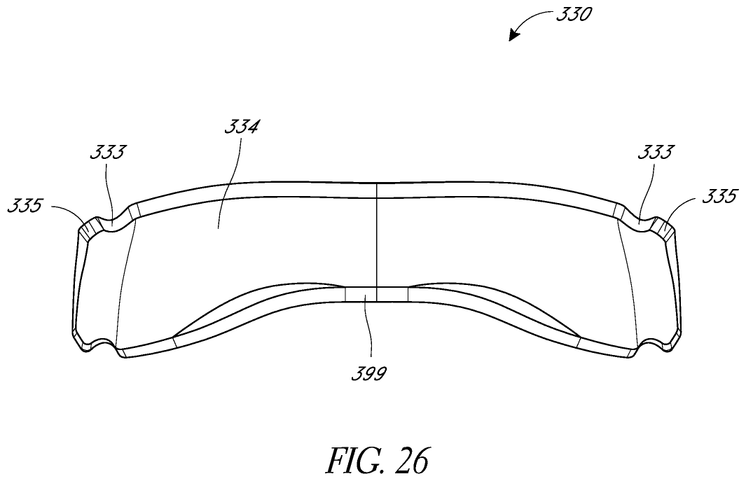

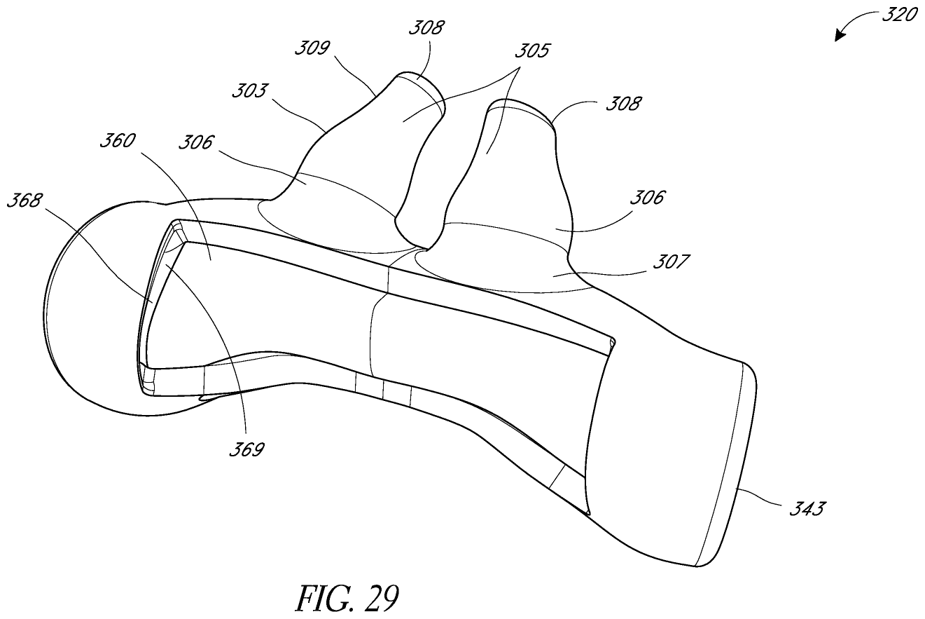

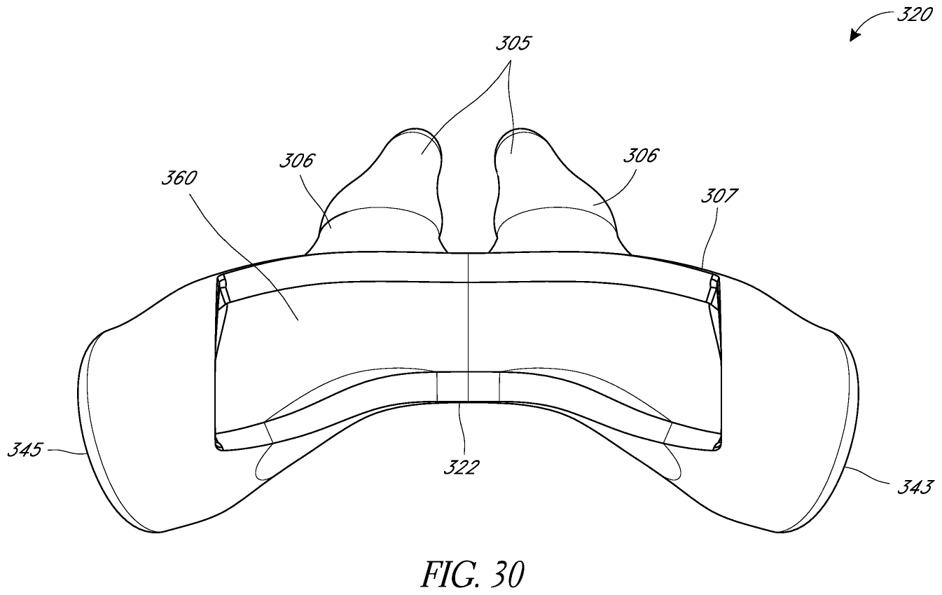

FIGS. 23-26 illustrate another embodiment of the clip 330 having a different attachment mechanism. The illustrated embodiment includes indentations 333 and winged portions 335. Each of the winged portions 335 extends from either side of the clip 330 and is designed to stick-out when the forward-facing side 332 of the clip 330 is attached to the user-facing side 314 of the body 313. In some embodiments, the entire forward-facing side 332 except for the winged portions 335 may be attached to or covered by the user-facing side 314 of the body 313. FIGS. 29-30 illustrate the nasal cannula portion 320 having the clip-receiving region 360 to which the clip 330 of FIGS. 23-26 can be attached. As shown in FIGS. 29-30, the clip-receiving region 360 includes a recessed window 368 to receive the clip 330 and one or more slots 369 extending from the recessed window 368 into the manifold 307. To couple the clip 330 to the clip-receiving region 360, each of the winged portions 335 may be inserted into the corresponding slot 369. The recessed window 368 may be shaped and sized to receive and conform to the clip 330.

The attachment mechanisms between the nasal cannula portion 320 and the clip 330 (e.g. as the push-fit domes 336/holes 362 or the winged portions 335/slots 369 or any other mechanisms described herein), may be advantageously durable enough to withstand multiple attachment/detachment cycle over the course of treatment, such that the clip 330 and the nasal cannula portion 320 have a sufficiently long useful life. Further, the attachment mechanisms may make a visible, audible or tactile feedback (e.g. clicking sound) when the connection is made, such that a user or clinician can know when the clip 330 and the nasal cannula portion 320 are properly engaged. The attachment mechanisms can also make a visible, audible, or tactile indication when the clip 330 and the nasal cannula portion are not properly attached. Also, when the nasal cannula portion 320 and the clip 330 are attached, the attachment mechanisms may allow decoupling of the nasal cannula portion 320 and the clip 330 only if a force exceeding a threshold level is exerted, to inhibit or prevent accidental detachment of the nasal cannula portion 320 and the clip 330. In some embodiments, the attachment mechanisms between the nasal cannula portion 320 and the clip 330 (e.g. as the push-fit domes 336/holes 362 or the winged portions 335/slots 369 or any other mechanisms described herein) can be engaged only when the nasal cannula portion 320 and the clip 330 (or the securement portion 310) are correctly oriented, such that they prevent incorrect assembly (e.g. upside-down) of the nasal cannula portion 320 and the securement portion 310. For example, the push-fit domes 336/holes 362 may have different shape at their upper end (e.g. round) than their lower end (e.g. straight).

In some embodiments, the clip 330 is permanently attached to the nasal cannula portion 320, and the attachment mechanisms described herein can be used to detachably attach the clip 330 to the securement portion 310. In such arrangements, the corresponding structures, the push-fit holes 362 or the slots 369 may be positioned on the securement portion 310 instead of the nasal cannula portion 320.

In some embodiments, the clip 330 is constructed from a rigid material, and the clip 330 may be more rigid than at least a portion of the nasal cannula portion 320, and/or at least a portion of the securement portion 310. When the securement portion 310 and the nasal cannula portion 320 is attached using the clip 330, the clip 330 can provide strength and stability to the patient interface 300 and prevent undesired collapsing of the patient interface. In some embodiments, the clip 330 can be or include a component formed of a material that has increased stiffness relative to the silicone or other material(s) forming the nasal cannula portion 320.

Further the clip 330 may span a significant portion of the width of the nasal cannula portion 320 to provide structural strength to the nasal cannula portion 320. In some embodiments, the clip 330 is the interface between the securement portion 310 and the nasal cannula portion 320, and the rigid clip 330 can inhibit or prevent forces exerted by the bridging portions 315 or facial pads 339 of the securement portion 310 from deforming or moving the nasal cannula portion 320. If the clip 330 spans a wider portion of the width of the nasal cannula portion 320, the clip 330 may provide more structural support. In some embodiments, the clip 330 may span the entire or substantially the entire width of the nasal cannula portion 320. In some embodiments, the clip 330 may span at least 15%, 20%, 30%, 50%, 60%, 70% or 80% of the width of the nasal cannula portion 320.

In some embodiments, the patient interface 300 may further include an additional coupling component to further secure the securement portion 310 and the nasal cannula portion 320. For example, the patient interface 300 may include an additional clip, pin, or bands to further secure the securement portion 310, and they can be further applied to the patient interface 300 after the securement portion 310 and the nasal cannula portion 320 are coupled using the clip 330 as described herein, such that undesired decoupling of the securement portion 310 and the nasal cannula portion 320 is prevented. The additional coupling component (e.g. a clip, a pin, a band) may be removable, such that they are removed before decoupling the securement portion 310 and the nasal cannula portion 320.

### Nasal Cannula Portion

FIGS. 27-30 illustrates the nasal cannula portion 320. As shown in FIGS. 1-11, the nasal cannula portion 320 may have the at least one nasal prong 305, such as a pair of prongs 305, extending from the manifold 307. The nasal prongs 305 can form a seal with nares of a user's nose and allow gas flow from and to the user. The nasal prongs 305 of the patient interface shown and described herein are shaped for sealing in a patient's nares. In some embodiments, the nasal prongs are shaped for sealing in an infant's nares. However, it should be appreciated that the prongs may be suitable for any patient population with similar nares geometry, and that the prongs may be provided in different absolute sizes for various patient populations. The prongs 305 can be shaped and formed to minimise tissue compression and kinking during insertion into a patient's nares. The prongs 305 are further described elsewhere herein.

In the illustrated embodiment, the manifold 307 defines a first opening 343 and a second opening 345 which can receive the interface tubes and allow gas flow from and to the patient via the manifold 307 and the nasal prongs 305. In some configurations, the first opening 343 is the gas inlet of the manifold 307 and the second opening 345 is the gas outlet of the manifold 307. In some configurations, the second opening 345 is the gas inlet of the manifold 307 and the first opening 343 is the gas outlet of the manifold 307. The first opening 343 and the second opening 345 may be in fluid communication with each other via the manifold 307. Except for the first and second openings 343, 345, and the lumens of the nasal prongs 305, the manifold 307 may be sealed. In some embodiments, the manifold 307 does not include a plurality of vent holes for patient exhalation or bias flow exhaust.

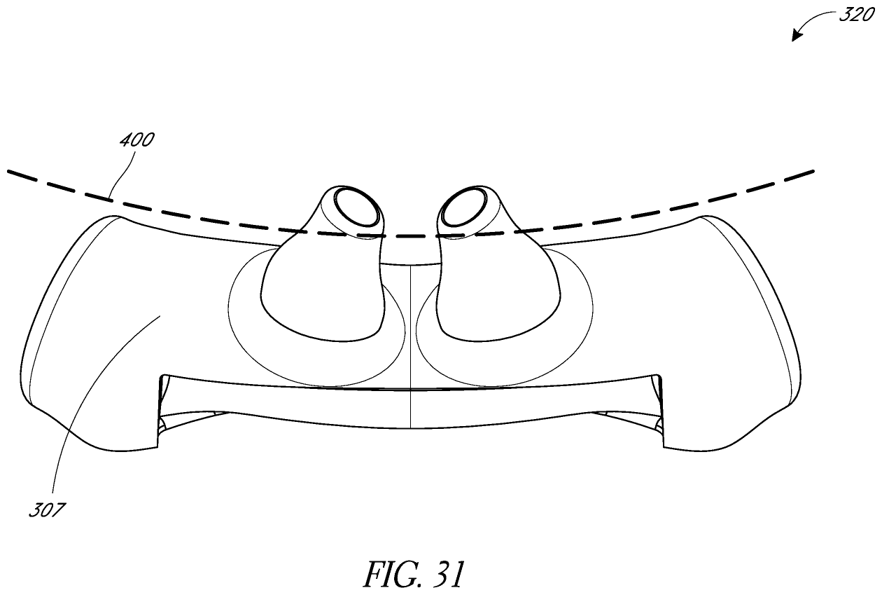

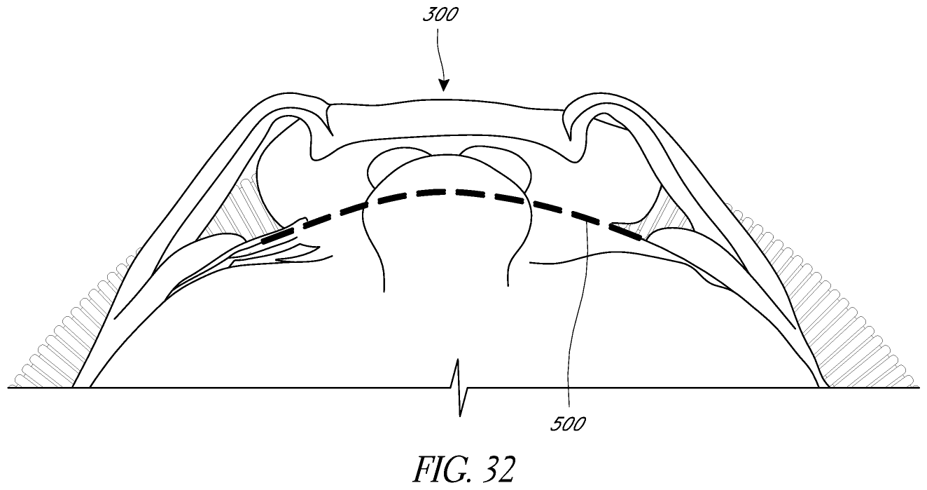

As shown in FIG. 31, when viewed above, the manifold 307 may have a generally arcuate profile as shown by a line 400. The curvature of this inner surface is shaped such that the patient interface 300 follows the contours of the user's face when in use. In some embodiments, as shown in FIG. 32, the curvature of the patient interface 300 at least substantially conforms to a curvature 500 of the user's face and the widest points on the patient interface 300 rest on the cheeks of the user.

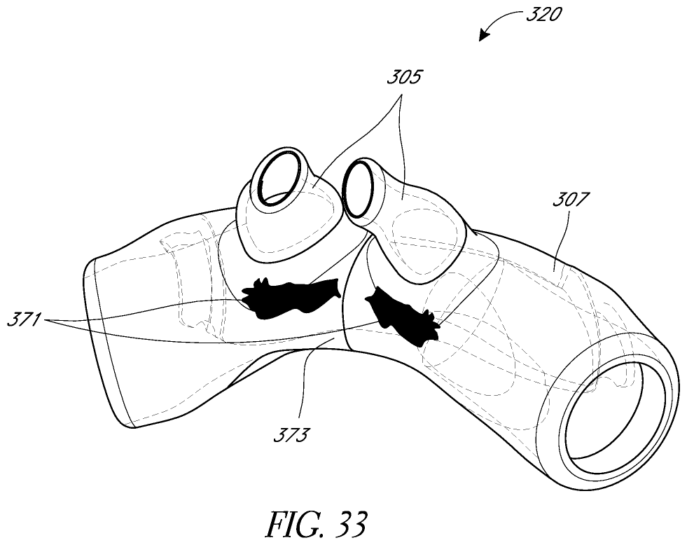

The manifold 307 may have varying thicknesses at different regions. For example, the clip-receiving region 360 may have a greater thickness than the rest of the manifold 307, such that the front side of the manifold 307 can withstand the attachment/detachment of the securement portion and strengthen the manifold 307 when the securement portion 310 is not attached. On the other hand, as shown in FIG. 33, an inner, patient-facing side 373 of the manifold 307 may be thinner, softer, and more flexible to conform over the user's philtrum and face with minimal skin damage. Further, the softer region of the manifold 307 may be fit snug against the user's philtrum in use, and allow the manifold 307 to have a larger region that can be in contact with the user, which can result in better distribution of pressure.

In the illustrated embodiment of FIG. 33, the patient-facing side 373 of the manifold 307 includes thinned regions 371 adjacent the prongs 305. The thinned regions 371 may be located under the prongs 305 and support the prongs and ensure that the prongs 305 collapse into the manifold 307 by the thinned regions 371 being folded, rather than kinking. The thinned regions 371 may be adjacent the softer region of the patient-facing side 373 which was described herein.

### Lower Curved Profile

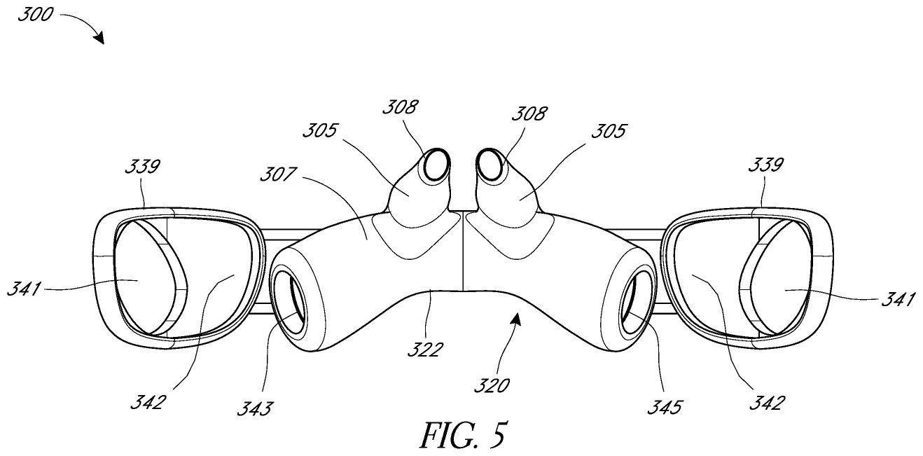

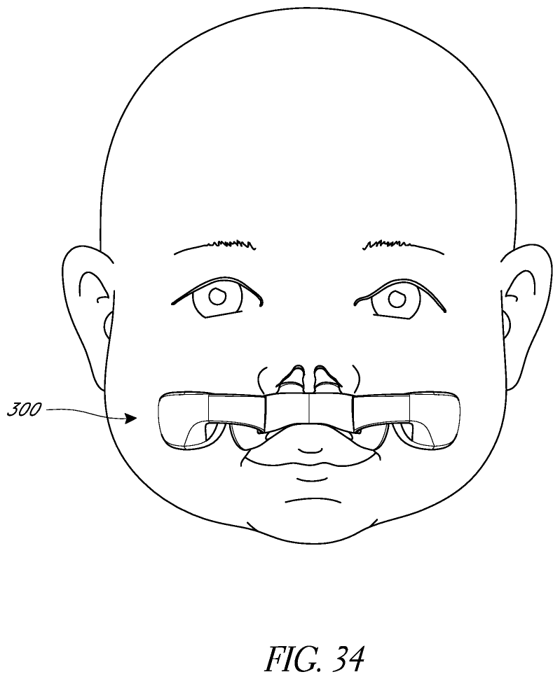

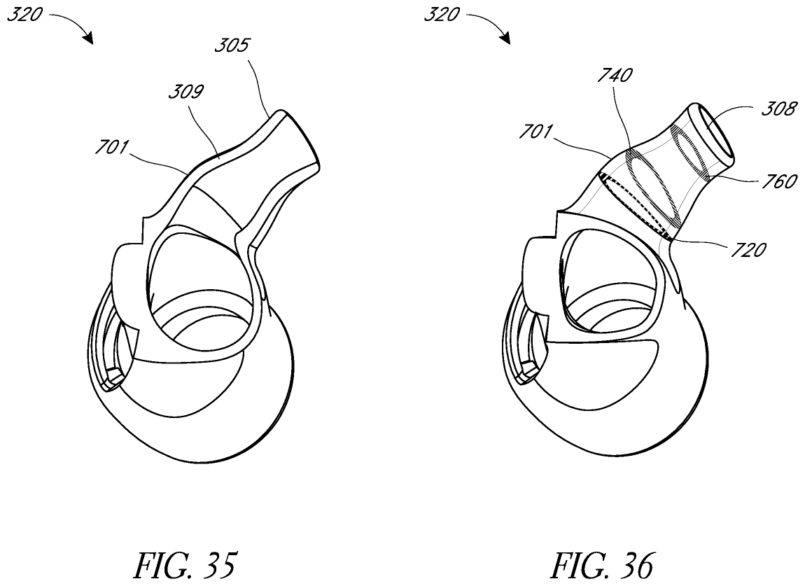

FIGS. 5, 10, 14, 15, 17-18, 20, 21, 25, 26, 28, 30 illustrate at least one or more of the followings: a lower edge 312 of the body 313 of the securement portion 310; a lower edge 399 of the clip 330; and a lower edge 322 of the nasal cannula portion 320. As shown in these figures, the lower edge 312 of the securement portion 310, the lower edge 399 of the clip 330, and the lower edge 322 of the nasal cannula portion 320 may be contoured upward. In some embodiments, the securement portion 310, the clip 330, and the nasal cannula portion 320 have at least partially conforming lower edge, and the entire patient interface 300 may define a lower edge at least partially curved upward, as shown in FIG. 35. As shown in FIG. 34, the upwardly curved lower edge of the patient interface 300 can allow the user's mouth to be exposed even when the user is wearing the patient interface 300, for the comfort of the user. If the user is an infant, a pacifier may be used or the user can be breastfed during the treatment. Further, an orogastric tube may be introduced to the patient, and the mouth of the user would be able to be cleaned easily.

Further, the curved profile of the lower edges of the body 313, the clip 330, and the nasal cannula portion 320 can indicate the orientation of these components, and may help a user or a clinician orient the components during assembling or disassembling the patient interface 300, thereby reducing the chance of error of assembling components upside down.

### Nasal Prongs

As shown in FIGS. 27 and 29, each of the nasal prongs 305 has the gas inlet 306 for fluid communication with a supply of breathable gas. Each of the nasal prongs 305 has the gas outlet 308 configured to direct a flow of gas towards nares of the user's nose. Each of the nasal prongs 305 further includes an exterior surface 303. At least a portion of the exterior surface 303 is a sealing surface 309. The sealing surface 309 is configured to seal the nares of the user's nose. In one embodiment, the sealing surface 309 is configured to seal with the nasal valve of the user's nose. In another embodiment, the sealing surface 309 is configured to seal between the nares entry and the nasal valve.

In some embodiments, the sealing region 309 is, or comprises, a tapered region with the cross-section of the exterior surface 303 of the prong 305 near the gas inlet 306 being larger than the cross-section of the exterior surface 303 of the prong 305 near the gas outlet 308. In the embodiment shown, the sealing region 309 tapers from about 5 mm to about 4 mm, for example. The measurements of the prong 305 (5 mm and 4 mm) refer to an equivalent diameter of a circle. The equivalent diameter may be a diameter calculated from the perimeter of the prong 305 (circular and ovate prongs may have the same equivalent diameter) or it may be the largest width dimension of the prong 305 (circular and ovate prongs may have a different equivalent diameter). The tapered sealing region 309 allows the patient interface 300 to be used for a patient having a nare size that is anywhere between about 4 mm and about 5 mm. For example, if a patient's nare size is 4.5 mm, the prongs 305 are inserted in the nares such that the section of the sealing region 309 having a diameter of 4.5 mm seals with their nares.

The prong sealing region 309 may have different dimensions and/or different amounts of taper. The length range for the sealing region may be between about 1 mm to about 10 mm. The length of the sealing region may be about 1 mm, 2 mm, 3 mm, 4 mm, 5 mm, 6 mm, 7 mm, 8 mm, 9 mm, or 10 mm. The diameter of the sealing region may be between about 2 mm and about 10 mm. The diameter of the sealing range may be about 2 mm, 3 mm, 4 mm, 5 mm, 6 mm, 7 mm, 8 mm, 9 mm, or 10 mm.

The ratio of the taper from the wider portion to the narrower portion may be about 1.1:1, 1.2:1, 1.3:1, 1.4:1, 1.5:1, 1.6:1, 1.7:1, 1.8:1, 1.9:1, or 2:1.

For example, the sealing region may taper from 10 mm to 9 mm, 9 mm to 8 mm, 8 mm to 7 mm, 7 mm to 6 mm, 6 mm to 5 mm, 5 mm to 4 mm, or 4 mm to 3 mm, 3 mm to 2 mm, for example. The taper may be steeper, for example, the prong sealing region may taper from 10 mm to 8 mm, 9 mm to 7 mm, 8 mm to 6 mm, 7 mm to 5 mm, 6 mm to 4 mm, 5 mm to 3 mm, or 4 mm to 2 mm, for example. The taper may be less steep, for example, the prong sealing region may taper from 10 mm to 9.5 mm, 9.5 mm to 9 mm, 9 mm to 8.5 mm, 8.5 mm to 8 mm, 8 mm to 7.5 mm, 7.5 mm to 7 mm, 7 mm to 6.5 mm, 6.5 mm to 6 mm, 6 mm to 5.5 mm, 5.5 mm to 5 mm, 5 mm to 4.5 mm, 4.5 mm to 4 mm, or 4 mm to 3.5 mm, 3.5 mm to 3 mm, 3 mm to 2.5 mm, or 2.5 mm to 2 mm for example.

Different sizes of the patient interface 300 and the nasal cannula portion 320 may be provided, for which different prong sealing region dimensions may be utilized.

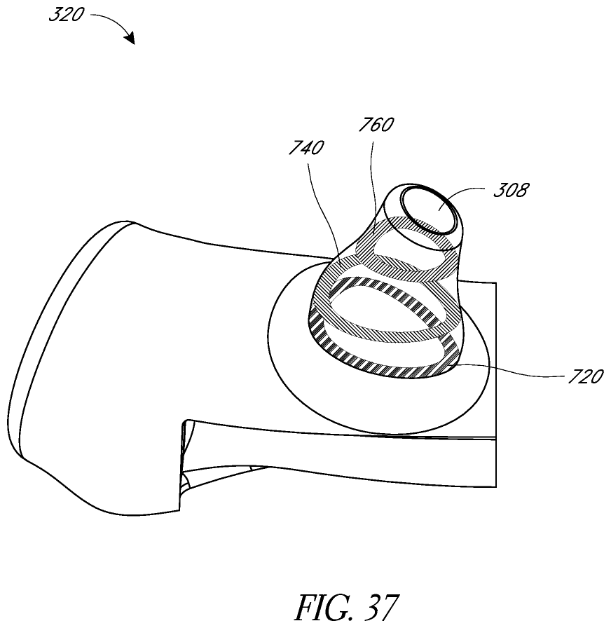

FIG. 35 illustrates a cross-section of the nasal cannula portion 320 along the length of the prong 305. FIG. 36 illustrates a cross-section of the nasal cannula portion 320 along the portion between two prongs 305, showing the prong 305 in a partial see-through view. FIG. 37 illustrates a top view of a portion of the nasal cannula portion 320, showing the prong 305 in a partial see-through view. In the illustrated embodiment, the sealing region 309 extends from the lower sealing plane 740 to the upper sealing plane 760, and the prong 305 defines a rounded corner 701 at a lower end of the sealing region 309 adjacent the lower sealing plane 740. Even though the sealing region 309 taper, the ratio of taper of the prong 305 changes abruptly adjacent the lower sealing plane 740 and the taper may be steeper above the lower sealing plane 740 than below the lower sealing plane 740, thereby defining the rounded corner 701. For example, the ratio of the taper from the lower sealing plane 740 to the upper sealing plane 760 may be greater than the taper from the base plane 720 to the lower sealing plane 740.

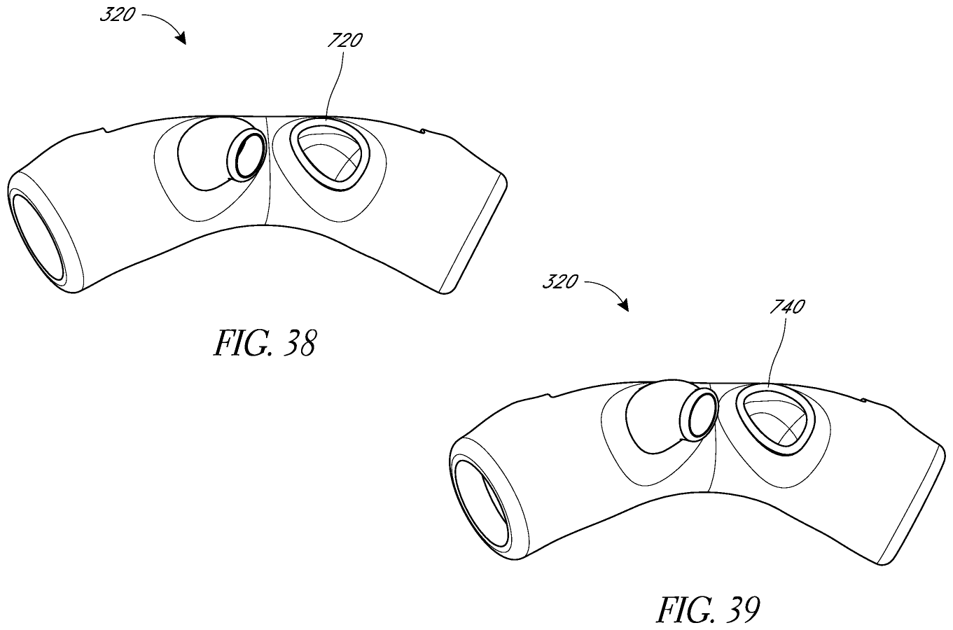

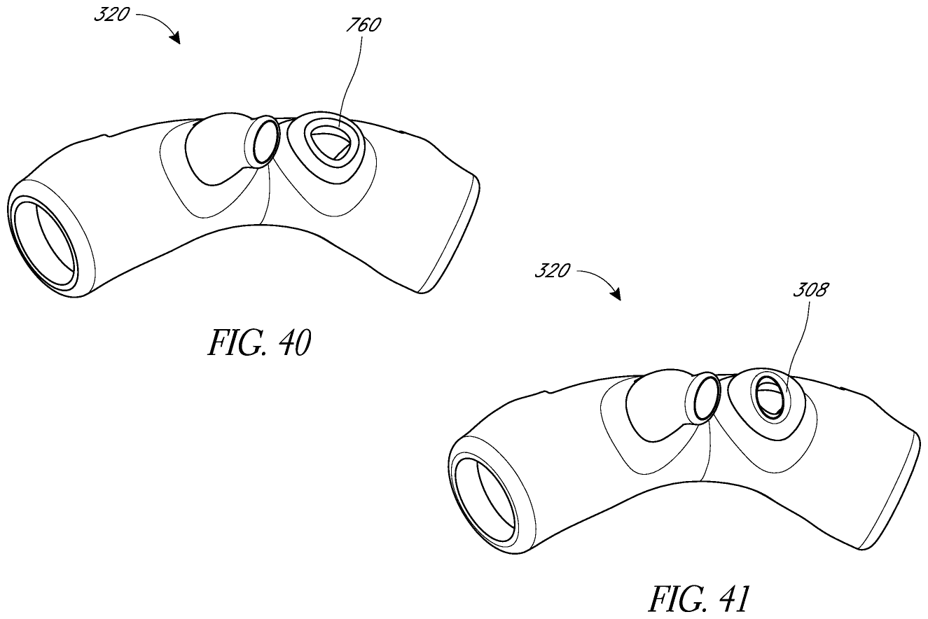

FIGS. 38-41 illustrate cross-sections of the prong 305 along the base plane 720, the lower sealing plane 740, the upper sealing plane 760, and the prong outlet 308, respectively. In the illustrated embodiment, the prong 305 has similar diameters at the base plane 720 and at the lower sealing plane 740, while the diameter decreases significantly from the lower sealing plane 740 to the upper sealing plane 760. In some embodiments, the prong 305 may not taper substantially from the base plane to the lower sealing plane. In some embodiments, the ratio of the taper from the base plane to the lower sealing plane may be 1:1, 1.01:1, 1.03:1, 1.05:1, 1.1:1 or 1.5:1.

The presence of the rounded corner 701 may limit further insertion of the prongs 305 into the nares past the rounded corner 701, and even if insertion of the prongs 305 were forced past the rounded corner 701, this would not improve the seal between the prongs 305 and the nares. Accordingly, the rounded corner 701 may provide a visual indicator to a user or a clinician that a larger prong size may be needed to have a desired seal in the patient's nares if the prongs 305 can be inserted into the nares past the rounded corner 701. This can be a useful indication as when the nasal cannula portion 320 is in use on an infant patient, the patient is not able to give reliable feedback on whether the interface is too big or too small. In some embodiments, to make this size indicator at the rounded corner 701 clearer to a user or a clinician, a line, a ridge or another mark may be used as a visual indicator of the desired maximum insertion depth at or near the rounded corner to show if a different prong size should be used.

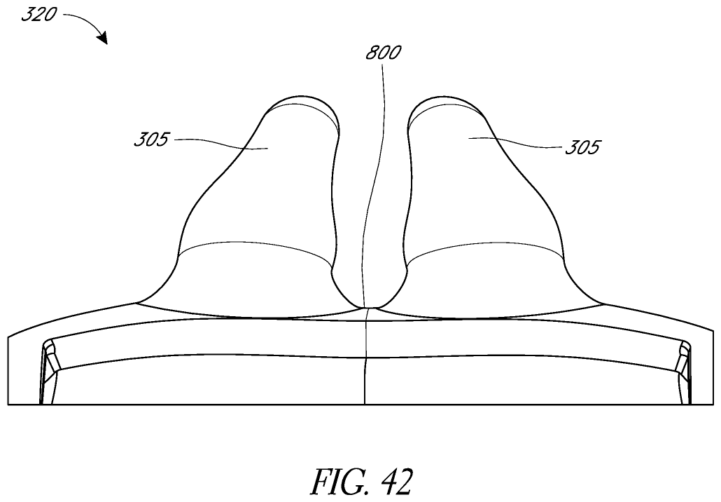

In some embodiments, the nasal prong 305 is shaped to avoid contact with the septum of a user at the base of a user's nose. With reference to FIG. 42, the nasal cannula portion 320 has two nasal prongs 305, where the two prongs 305 define a recess 800 between the prongs 305 to avoid contact with the septum. In this example embodiment, the recess 800 is shown as a scalloped recess. It should be appreciated by one of skill in the art that any suitable shape of recess may be used. Preferably, the shape is configured to reduce contact between the interface and the patient's septum, or reduces pressure on the patient's septum as compared to a horizontal or straight body connection between the bases of the prongs. The recess has a smooth transition between each of the nasal prongs 305 and the recess 800 and may be shaped like the bottom of a tear drop. This curvature can ensure septum spacing with the cannula. This spacing allows the patient interface to seal with the patient's nares without the portion between the prongs touching or impinging upon the patient's septum. This reduces contact pressure on the septum and possible septum damage. However, it should be noted that in some situations, the portion of the patient interface between the nasal prongs may contact the septum. In this situation, the recess would conform to the patient's septum and ensure only a small amount of pressure is applied to the septum. The recess conforms by using a thin wall thickness compared to other regions of the base section or prongs of the patient interface, which reduces the pressure the recess applies to the septum. As well as this function, the thinness of this region may also allow the prongs 305 to have some movement in the inward direction (towards each other) and/or the outward direction (away from each other) which may help fitting the prongs 305 to some patients.

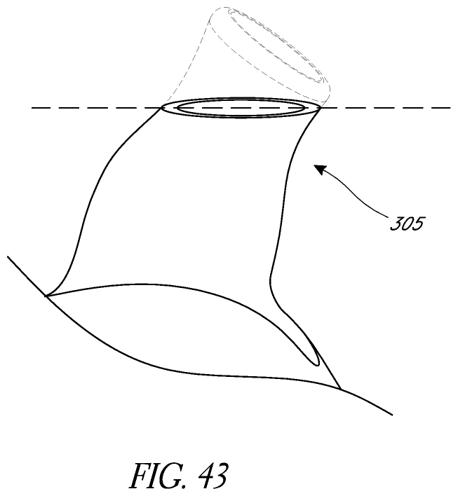

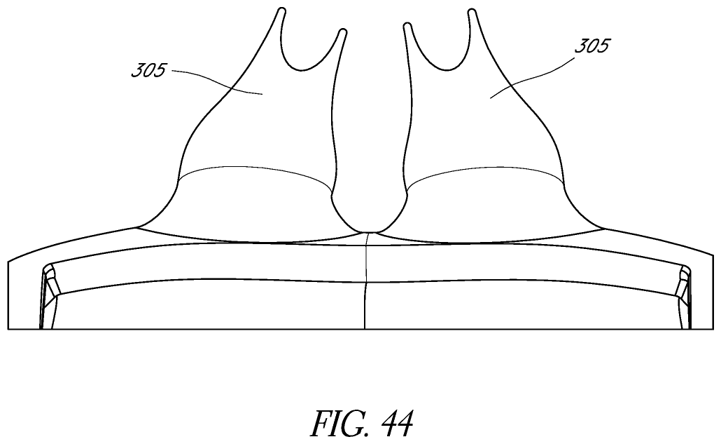

Top ends of the prongs 305 adjacent the prong outset 308 may be shaped to reduce the resistance to flow of the prongs 305. For example, as shown in FIG. 43, the prongs 305 may be cut off horizontally or at an angle. This could lower the resistance in the prong. Additionally, or alternatively, when viewing the prongs 305 from above the interface 300 in a same or similar perspective of FIG. 42, the prong outlet 308 may have a U-shape, as shown in FIG. 44. These features may provide a lower resistance to flow in use, and/or prevent accidental sealing of the prong outlet 308, which may cause a user or a clinician to falsely determine that the CPAP therapy is delivered to the user.

### Interface Conduits Management

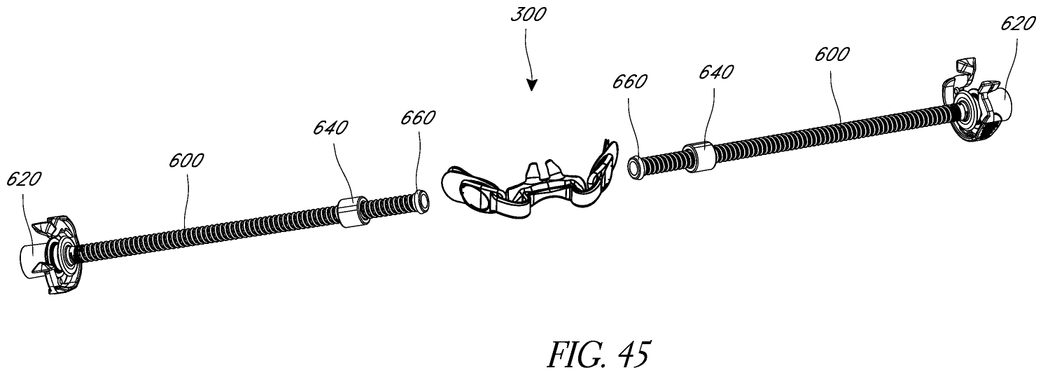

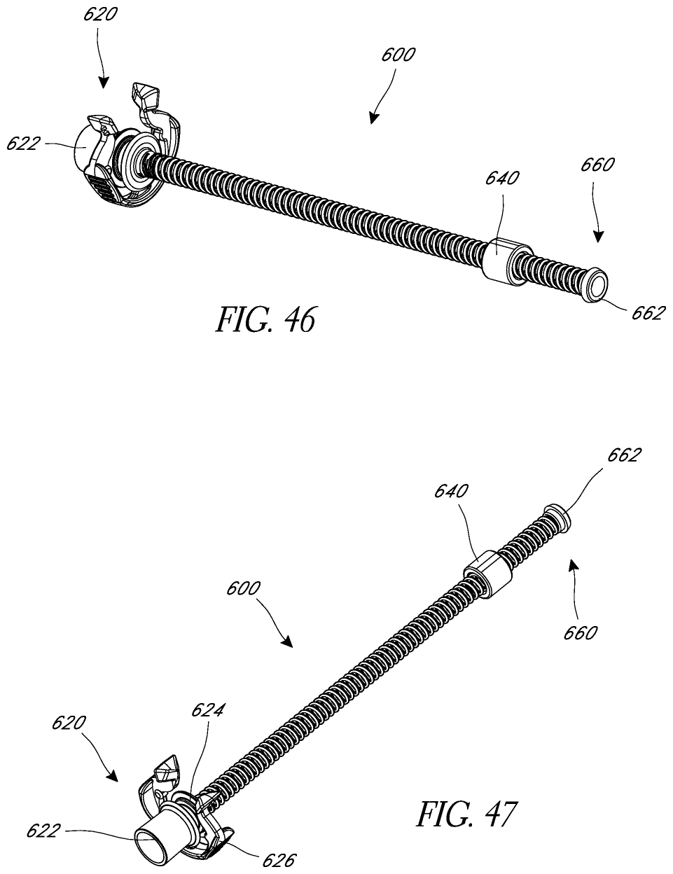

Each respiratory interface tube of the present disclosure has a respiratory component end (expiratory/inspiratory side, such as for connection to the tubes 201a and 201b) that connects to a CPAP therapy delivering device and a patient interface end that, in this present disclosure, connects to one side of the patient interface 300, for example to the first opening 343 and the second opening 345 of the nasal cannula portion 320. FIG. 45 illustrates the patient interface 300 and interface tubes or conduits 600, and FIGS. 46-47 illustrate the perspective views of the interface tubes or conduits 600. In the illustrated embodiment, each of the interface tubes or conduits 600 includes a patient interface end 660, a respiratory component end 620, and an engagement portion 640.

The patient interface end 660 may be rigid compared to an adjacent section of the tube. Alternatively, the patient interface end 660 may be soft, or less rigid, compared to an adjacent section of the tube. For example, the patient interface end 660 may comprise a flexible, supple, or compliant material. The patient interface end 660 of the tube 600 may be a separate component to the tube 600, or may be an integral part of the tube 600. The patient interface end 660 of the tube 600 may be an overmoulded component, such as an overmoulded connector 662. The connector 662 may be or comprise a silicone or elastomer material. An embodiment having a soft patient interface end increases patient comfort, for example, when the patient's face is on the patient interface end 660, which may occur when the patient is lying on their side or front.

The respiratory component end 620 of the tube 600 may include a connector 622 for the connection to the respiratory component, such as tubes 201a and 201b. The connector 622 may be overmoulded to the tube 600 at the respiratory component end 620. The connector 622 may be or comprise a silicone or elastomer material. The connector 622 at the respiratory component end 620 of the tube 600 may be suitable for receiving a clip 626. For example, the connector 622 may have a recess or a ridge 624 for holding the clip 626 in place. The clip 626 may be rotatable while sitting in the recess 624 and may be used to clip to bed sheets or clothing to provide additional securement of the patient interface 300 and the tubes 600.

In some occasions, it may be desirable that there is some way to hold and manage the interface tubes 600 that connect to the nasal cannula portion 320. In such arrangements, the tubes 600 may be flexible enough to promote tube movement and fixation by a clinician or a user. Being able to move the tubes 600 and fix as desired may allow more comfort for the patient, for example side sleeping.

In some embodiments, the bridging portion 315 of the securement portion 310 may have cutout or recessed regions for receiving the tubes 600. The cutout or recessed regions may further include hook or loop material for attaching to a corresponding material on the tubes 600. The cutout regions may be located at any suitable location along the bridging portion 315 or the facial pad 339. In some embodiments, there may be one or more cutout or recessed regions on the bridging portion 315 or the facial pad 339. In some embodiments, there may be a cutout or recessed region having hook or loop on the forward-facing side (non-patient facing side) of each facial pad 339 or the bridging portion 315. In some embodiments, the hook or loop material may be overmoulded onto the bridging portions 315 or the facial pad 339, or attached by adhesive.

In some embodiments, the outward facing and patient facing hook or loop pieces of material may be formed as a single piece to form a double-sided hook or loop material, which can be overmoulded into a cutout or opening in the facial pad 339, as described with regard to FIGS. 13-15 elsewhere. The use of the double-sided hook or loop material may be advantageous, since it can be overmoulded more easily to the securement portion 310 than separate pieces of hook or loop on each side. Further, the facial pad 339 may be more flexible when a single double-sided piece of hook or loop material is used, as opposed to multiple pieces of hook or loop materials, thereby conforming to the user's face better.

The tube 600 may have a strip of hook or loop material to be attached with a corresponding material on the patient interface 300. For example, the strip of hook or loop may be wrapped around the outside of the tube 600 or attached to a part of the tube 600. In some embodiments, the tube 600 may include the engagement portion 640 where the strip of hook or loop or any other attachment mechanism is attached to. The engagement portion 640 may be wrapped or moulded or placed around the outside of the tube 600. The piece of loop material may attach to the corresponding hook material on the bridging portions 315 of the patient interface 300 to secure the tube 600. Alternatively, or additionally there may be an overmoulded portion on the tube where part of this portion comprises loop material.

In some embodiments, magnets may be used to hold the conduit or tube 600 on the patient interface 300. For example, there may be a magnet on the facial pad 339 or the bridging portion 315, or placed on a piece of material that wraps around the tube 600 or overmoulded into the tube 600. This magnet can interact with a corresponding ferromagnetic material on the other component to secure the tube 600 and interface 300. Any other suitable detachable coupling mechanisms, such as mechanism described elsewhere herein regarding attachment of the securement portion 310 and the nasal cannula portion 320, may be used to attach the tubes 600 to the securement portion 310.

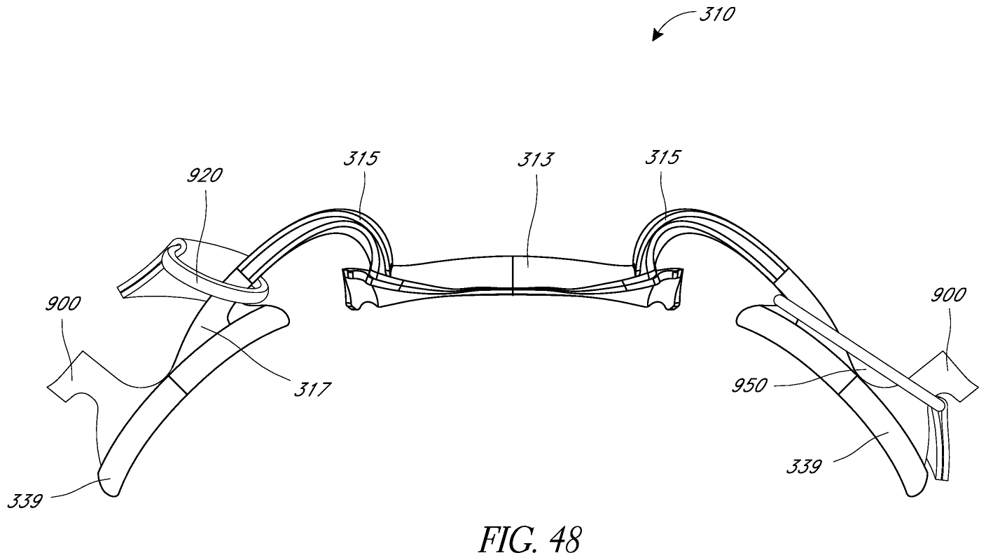

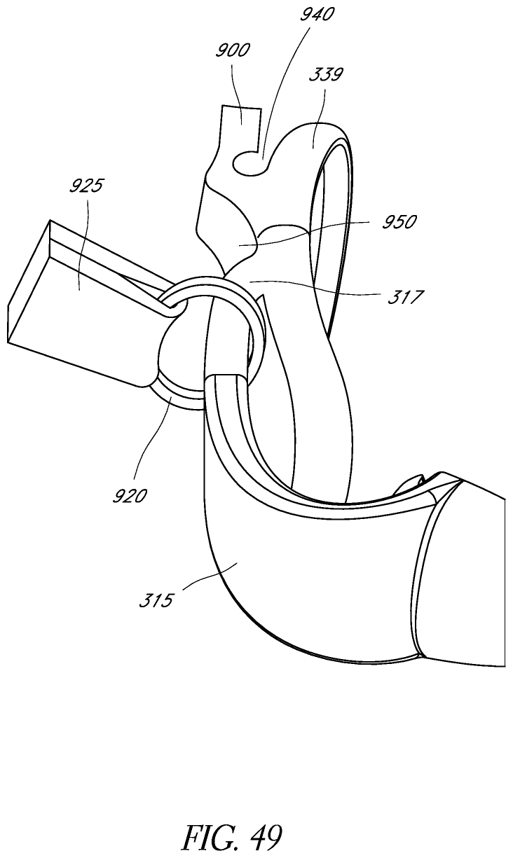

In some embodiments, an elastomeric band may be used to hold the tube or conduit 600 on the securement portion 310. FIGS. 48-49 illustrate an embodiment of the securement portion 310 having an elastomeric band 920. The band 920 may be located around the bridging portion 315 and positioned adjacent the facial pad 339. In some embodiments, the band 920 may be located adjacent the base or connecting portion 317. In the illustrated embodiment, the facial pad 339 includes a raised portion 900 which defines a notch 940 for receiving the band 920. To fix the tube or conduit 600, the band 920 may be pulled and received in the notch 940. As shown in FIGS. 45 and 46, in some embodiments, the facial pad 339 include a groove 950 at a forward-facing side (non-patient facing side) where a portion of the conduit or tube 600 can be received. The groove 950 can be at least partially defined by the raised portion 900 and the base or connecting portion 317, and the tube or conduit 600 can be fixed in a space defined by the band 920 and the groove 950. As shown in FIG. 49, the band 920 may include a tab 925 connected to the band 920, such that the band 920 can be grabbed and pulled more easily. In some embodiments, the elastomeric band 920 are moulded in a square or rectangular profile to distribute the forces during conduit securement more evenly along the contact regions of the elastomeric band 920.

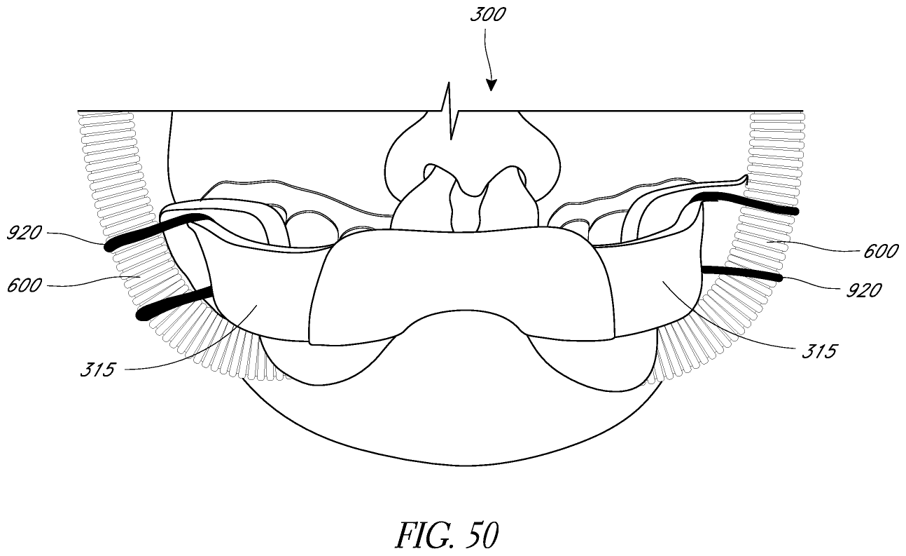

FIG. 50 illustrates the patient interface 300 applied on a user. In the illustrated embodiment, the tubes 600 are connected to the interface 300. The tubes 600 may be fixed using the elastomeric band 920 extending around the bridging portion 315 and the tube 600.

### Terminology

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes, or tends toward, a particular value, amount, or characteristic. For example, as the context may dictate, the term “generally linear” can mean something that departs from exactly parallel by less than or equal to 15°.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

Reference to any prior art in this specification is not, and should not be taken as, an acknowledgement or any form of suggestion that that prior art forms part of the common general knowledge in the field of endeavor in any country in the world.

The invention may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

1. A patient interface, comprising:
a nasal interface comprising:
a manifold; and
one or more prongs extending from the manifold; and
a securement portion comprising;
a body; and
a pair of bridging portions, each of the pair of bridging portions extending from the body;
wherein the nasal interface and the securement portion are detachably coupled to each other.

2.-28. (canceled)

29. A patient interface, comprising:
a user interface portion; and
a securement portion comprising:
a body;
a pair of facial pads, each of the pair of facial pads configured to engage a face of a user on opposing sides of the body; and
a pair of bridging portions, each of the pair of bridging portions connecting the body and a respective one of the pair of facial pads;
wherein the user interface portion and the securement portion is detachably attached to each other.

30. The patient interface of claim 29, wherein the user interface portion is one selected from a group consisting of: a sealing cannula, a non-sealing cannula, a nasal mask, and an oronasal mask.

31. The patient interface of claim 29, wherein the user interface portion is detachably attached to the securement portion at the body of the securement portion.

32. The patient interface of claim 29, further comprising a connection mechanism which detachably attaches the user interface portion with the securement portion.

33.-35. (canceled)

36. The patient interface of claim 29, further comprising one or more tubes configured to be connected to a manifold of the user interface portion.

37. (canceled)

38. The patient interface of claim 36, wherein the one or more tubes are inspiratory or expiratory tubes.

39. (canceled)

40. The patient interface of claim 29, wherein the user interface portion has a thinner region at a patient facing side.

41. The patient interface of claim 29, further comprising a patch assembly coupled to each of the pair of facial pads for securing the pair of facial pads to the user.

42. The patient interface of claim 41, wherein the patch assembly comprises a hook or loop material.

43. The patient interface of claim 29, wherein the pair of bridging portions have a curved shape configured in use to urge the user interface portion toward nares of the user to create or maintain sealing engagement between the user interface portion and the nares of the user.

44. The patient interface of claim 43, wherein the pair of bridging portions are configured to roll in response to movement between the pair of facial pads and the body.

45. (canceled)

46. The patient interface of claim 29, wherein the securement portion comprises a component which can be detachably attached to a respiratory tube or a conduit.

47. (canceled)

48. The patient interface of claim 29, wherein the user interface portion has a manifold defining a first opening and a second opening to allow gas flow to and from the user via the manifold, wherein the first opening is a gas inlet and the second opening is a gas outlet, and the first opening and the second opening are in fluid communication with each other via the manifold.

49. The patient interface of claim 29, wherein the user interface portion is a nasal cannula comprising a manifold and one or more prongs extending from the manifold, and wherein an inner, user facing side of the manifold is thinner to conform over a philtrum of the user.

50. The patient interface of claim 29, wherein the user interface portion is a nasal cannula comprising a manifold and a pair of prongs, the manifold comprising thinned regions adjacent to the pair of prongs.

51. The patient interface of claim 29, wherein each of the pair of bridging portions are provided as elongate arms shaped to have a non-linear or curved path between a first end at a junction between the body and the bridging portion and a second end at a junction between each of the pair of bridging portions and a base of the associated facial pad.

52. The patient interface of claim 29, wherein each of the pair of facial pads are configured to secure to the face of the user directly or indirectly by an external force, using one or more of an adhesive connection, a headgear arrangement, or a hook or loop connection.

53. The patient interface of claim 29, wherein each of the pair of bridging portions is attached to the body separately.

54. The patient interface of claim 29, wherein each of the pair of bridging portions is attached to a front of the securement portion.
